(19) United States
(12) Patent Application Publication
(10) Pub. No.: US 2023/0419224 A1
(43) Pub. Date: Dec. 28, 2023

(54) NETWORKED COMPUTING DEVICES

(71) Applicant: Express Scripts Strategic Development, Inc., St. Louis, MO (US)

(72) Inventors: Jayamalini Subramaniam, Chesterfield, MO (US); Antonio Brkovic, Nashville`, TN (US)

(21) Appl. No.: 18/367,539

(22) Filed: Sep. 13, 2023

Related U.S. Application Data
(63) Continuation-in-part of application No. 17/213,969, filed on Mar. 26, 2021.
Application No. 17/213,969 is a continuation of application No. 16/596,110, filed on Oct. 8, 2019, now abandoned.
Application No. 16/596,110 is a continuation-in-part of application No. 15/273,599, filed on Sep. 22, 2016, now abandoned.
(60) Provisional application No. 62/221,720, filed on Sep. 22, 2015.

(51) Int. Cl. G06Q 10/0635; G06Q 30/018; G16H 20/10

(52) U.S. Cl. CPC G06Q 10/0635; G06Q 30/018; G16H 20/10

(57) ABSTRACT

A governmental regulatory computing device, a user computing device, a participant computing device, and an analysis computing device communicate via a network. The analysis computing device determines a risk associated with at least one of a drug and a drug manufacturer based on data from the governmental regulatory computing device and a predicted demand from the participant computing device. The user computing device: requests and receives the risk from the analysis computing device; generates a report regarding the at least one of the drug and the drug manufacturer and include the risk in the report: and displays the report including the risk and the at least one of the drug and the drug manufacturer on a display. The analysis computing device, based on the risk associated with at least one of the drug and the drug manufacturer, selectively automatically orders a quantity of the drug from the drug manufacturer.

### CROSS-REFERENCE TO RELATED APPLICATIONS

This application is continuation-in-part of U.S. application Ser. No. 17/213,969, filed Mar. 26, 2021, which is a continuation of U.S. application Ser. No. 16/596,110, filed Oct. 8, 2019, which is a continuation-in-part of U.S. application Ser. No. 15/273,599, filed Sep. 22, 2016, which claims the benefit of U.S. Provisional Application No. 62/221,720, filed Sep. 22, 2015. The entire disclosures of the above are hereby incorporated by reference.

### FIELD

The field relates to drug supply chains, and more particularly to assessing drug supply chains.

### BACKGROUND

Data and analytics are being used to tackle a number of different problems that businesses of varying sizes face. Often times, the low costs associated with data collection results in business having a large set of data. Through simple and sometimes quite sophisticated methodologies, these large data sets can be analyzed and used for a wide variety of purposes.

### SUMMARY

In a feature, a method of determining a risk of a vendor of a drug includes: by one or more processors, obtaining first level risks associated with the vendor of the drug; by the one or more processors, retrieving first predetermined weight values associated with the first level risks, respectively, from memory; by the one or more processors, applying the first predetermined weight values to the first level risks to obtain weighted first level risks, respectively; by the one or more processors, calculating second level risks associated with the vendor based on the weighted first level risks; by the one or more processors, retrieving second predetermined weight values associated with the second level risks, respectively, from memory; by the one or more processors, applying the second predetermined weight values to the second level risks to obtain weighted second level risks, respectively; by the one or more processors, calculating third level risks associated with the vendor based on the weighted second level risks; by the one or more processors, retrieving third predetermined weight values associated with the third level risks, respectively, from memory; by the one or more processors, applying the third predetermined weight values to the third level risks to obtain weighted third level risks, respectively; by the one or more processors, calculating an overall risk associated with the vendor based the weighted third level risks; and by the one or more processors, in response to receipt of user input for the overall risk for the vendor of the drug, displaying a visual indicator of the overall risk of the vendor of the drug on a display.

In further features, calculating the second level risks includes setting the second level risks equal to sums of ones of the weighted first level risks.

In further features, calculating the third level risks includes setting the third level risks equal to sums of ones of the weighted second level risks.

In further features, calculating the overall risk includes setting the overall risk equal to a sum of the weighted third level risks.

In further features, one of the first level risks is a regulatory compliance risk, and the one of the first predetermined weighting values associated with the regulatory compliance risk is greater than all of the other ones of the first predetermined weighting values.

In further features, obtaining the first level risks includes obtaining at least one of the first level risks from an electronic drug vendor query.

In further features, obtaining the first level risks includes storing at least one of the first level risks in response to user input regarding the at least one of the first level risks.

In further features, obtaining the first level risks includes retrieving at least one of the first level risks from a database.

In further features, the first level risks are each a value between 0 and 100, and the first predetermined weighting values are values between 0 and 100 percent.

In further features, the second level risks are each a value between 0 and 100, and the second predetermined weighting values are values between 0 and 100 percent.

In further features, the third level risks are each a value between 0 and 100, and the third predetermined weighting values are values between 0 and 100 percent.

In further features, the first level risks include at least 10 different first level risks associated with the vendor of the drug.

In further features, the second level risks include at least 5 different second level risks.

In further features, the third level risks include 2 different third level risks.

In a feature, a system for determining a risk of a vendor of a drug includes: memory including instructions; and one or more processors configured to execute the instructions to: obtain first level risks associated with the vendor of the drug; retrieve first predetermined weight values associated with the first level risks, respectively, from the memory; apply the first predetermined weight values to the first level risks to obtain weighted first level risks, respectively; calculate second level risks associated with the vendor based on the weighted first level risks; retrieve second predetermined weight values associated with the second level risks, respectively, from the memory; apply the second predetermined weight values to the second level risks to obtain weighted second level risks, respectively; calculate third level risks associated with the vendor based on the weighted second level risks; retrieve third predetermined weight values associated with the third level risks, respectively, from the memory; apply the third predetermined weight values to the third level risks to obtain weighted third level risks, respectively; calculate an overall risk associated with the vendor based the weighted third level risks; and in response to receipt of user input for the overall risk for the vendor of the drug, display a visual indicator of the overall risk of the vendor of the drug on a display.

In a feature, a method of determining a risk associated with a drug includes: by one or more processors, obtaining first level risks associated with the drug; by the one or more processors, retrieving first predetermined weight values associated with the first level risks, respectively, from memory; by the one or more processors, applying the first predetermined weight values to the first level risks to obtain weighted first level risks, respectively; by the one or more processors, calculating second level risks associated with the drug based on the weighted first level risks; by the one or more processors, retrieving second predetermined weight values associated with the second level risks, respectively, from memory; by the one or more processors, applying the second predetermined weight values to the second level risks to obtain weighted second level risks, respectively; by the one or more processors, calculating an overall risk associated with the drug based the weighted second level risks; and by the one or more processors, in response to receipt of user input for the overall risk for the drug, displaying a visual indicator of the overall risk for the drug on a display.

In further features, calculating the second level risks includes setting the second level risks equal to sums of ones of the weighted first level risks.

In further features, calculating the overall risk includes setting the overall risk equal to a sum of the weighted second level risks.

In further features, obtaining the first level risks includes obtaining at least one of the first level risks from an electronic drug vendor query.

In further features, obtaining the first level risks includes storing at least one of the first level risks in response to user input regarding the at least one of the first level risks.

In further features, obtaining the first level risks includes retrieving at least one of the first level risks from a database.

In further features, the first level risks are each a value between 0 and 100, and the first predetermined weighting values are values between 0 and 100 percent.

In further features, the second level risks are each a value between 0 and 100, and the second predetermined weighting values are values between 0 and 100 percent.

In further features, the first level risks include at least 10 different first level risks associated with the drug.

In further features, the second level risks include 4 different second level risks.

In a feature, a system for determining a risk for a drug includes: memory including instructions; and one or more processors configured to execute the instructions to: obtain first level risks associated with the drug; retrieve first predetermined weight values associated with the first level risks, respectively, from memory; apply the first predetermined weight values to the first level risks to obtain weighted first level risks, respectively; calculate second level risks associated with the drug based on the weighted first level risks; retrieve second predetermined weight values associated with the second level risks, respectively, from memory; apply the second predetermined weight values to the second level risks to obtain weighted second level risks, respectively; calculate an overall risk associated with the drug based the weighted second level risks; and by the one or more processors, in response to receipt of user input for the overall risk for the drug, display a visual indicator of the overall risk for the drug on a display.

In a feature, a method of determining a risk associated with a drug and a vendor of that drug includes: by one or more processors, obtaining first level risks associated with the vendor of the drug; by the one or more processors, retrieving first predetermined weight values associated with the first level risks associated with the vendor, respectively, from memory; by the one or more processors, applying the first predetermined weight values to the first level risks associated with the vendor to obtain weighted first level risks associated with the vendor, respectively; by the one or more processors, calculating second level risks associated with the vendor based on the weighted first level risks associated with the vendor; by the one or more processors, retrieving second predetermined weight values associated with the second level risks associated with the vendor, respectively, from memory; by the one or more processors, applying the second predetermined weight values to the second level risks associated with the vendor to obtain weighted second level risks associated with the vendor, respectively; by the one or more processors, calculating third level risks associated with the vendor based on the weighted second level risks associated with the vendor; by the one or more processors, retrieving third predetermined weight values associated with the third level risks associated with the vendor, respectively, from memory; by the one or more processors, applying the third predetermined weight values to the third level risks associated with the vendor to obtain weighted third level risks associated with the vendor, respectively; by the one or more processors, calculating a first overall risk associated with the vendor based the weighted third level risks associated with the vendor; by the one or more processors, obtaining first level risks associated with the drug; by the one or more processors, retrieving fourth predetermined weight values associated with the first level risks associated with the drug, respectively, from memory; by the one or more processors, applying the fourth predetermined weight values to the first level risks associated with the drug to obtain weighted first level risks associated with the drug, respectively; by the one or more processors, calculating second level risks associated with the drug based on the weighted first level risks; by the one or more processors, retrieving fifth predetermined weight values associated with the second level risks associated with the drug, respectively, from memory; by the one or more processors, applying the fifth predetermined weight values to the second level risks associated with the drug to obtain weighted second level risks associated with the drug, respectively; by the one or more processors, calculating a second overall risk associated with the drug based the weighted second level risks associated with the drug; by the one or more processors, applying the sixth and seventh predetermined weight values to the first and second overall risks to obtain weighted first and second overall risks, respectively; by the one or more processors, calculating a third overall risk associated with the vendor supplying the drug based on the weighted first and second overall risks; and by the one or more processors, in response to receipt of user input for the third overall risk, displaying a visual indicator of the third overall risk on a display.

In further features, calculating the third overall risk includes setting the third overall risk equal to a sum of the weighted first and second overall risks.

In a feature, a system includes: a network; a governmental regulatory computing device; a user computing device; a participant computing device; and an analysis computing device including memory and a processor configured to: communicate with the governmental regulatory computing device via the network; obtain, from the governmental regulatory computing device, data regarding at least one of a drug manufacturer that is subject to an exclusion order and a drug that is subject to an exclusion order; and determine a risk associated with at least one of the drug and the drug manufacturer based on the data obtained from the governmental regulatory computing device, where the participant computing device is configured to transmit a predicted demand for the at least one of the drug and the drug manufacturer to the analysis computing device via the network, where the analysis computing device is configured to determine the risk associated with the at least one of the drug and the drug manufacturer further based on the predicted demand for the at least one of the drug and the drug manufacturer, where the user computing device is configured to transmit a query to the analysis computing device over the network, the query requesting the risk associated with the at least one of the drug and the drug manufacturer, where the analysis computing device is configured to transmit the risk of the at least one of the drug and the drug manufacturer to the user computing device via the network, where the user computing device is configured to: generate a report regarding the at least one of the drug and the drug manufacturer and include the risk in the report: and display the report including the risk and the at least one of the drug and the drug manufacturer on a display, and where the analysis computing device is further configured to, based on the risk, selectively automatically order a quantity of the drug from the drug manufacturer.

In a feature, a method of ordering a drug from a vendor of the drug includes: by one or more processors, using a first application programming interface (API), obtaining first level risk values associated with the vendor of the drug from two or more external data sources, the first level risk values representing real world quantities used to evaluate risk; by the one or more processors, retrieving first predetermined weight values associated with the first level risk values, respectively, from a SQL database; by the one or more processors, applying the first predetermined weight values to the first level risk values to obtain weighted first level risk values, respectively; by the one or more processors, calculating second level risk values associated with the vendor based on the weighted first level risk values; by the one or more processors, retrieving second predetermined weight values associated with the second level risk values, respectively, from the SQL database; by the one or more processors, applying the second predetermined weight values to the second level risk values to obtain weighted second level risk values, respectively; by the one or more processors, calculating third level risk values associated with the vendor based on the weighted second level risk values; by the one or more processors, retrieving third predetermined weight values associated with the third level risk values, respectively, from the SQL database; by the one or more processors, applying the third predetermined weight values to the third level risk values to obtain weighted third level risk values, respectively; by the one or more processors, calculating an overall risk value associated with the vendor based the weighted third level risk values; by the one or more processors, in response to receipt of user input for the overall risk value for the vendor of the drug, displaying a visual indicator of the overall risk value of the vendor of the drug on a display, by the one or more processors, changing one of the first, second, and third level risk values and recalculating the overall risk value using the changed one of the first, second, and third level risk values via retrieval using a second API; by the one or more processors, visually indicating on the display all of the first, second, and third level risk values that drop below a first threshold in response to the changing of the one of the first, second, and third level risk values; by the one or more processors, transmitting an electronic alert to an associated device when one of the first, second, and third level risk values exceeds a second threshold; by the one or more processors, visually changing a risk indicator as at least one of the first level, second level, third level, and overall risk values increases; and by the one or more processors, based on the overall risk value, selectively ordering a quantity of the drug from the vendor.

In further features, calculating the second level risk values includes setting the second level risk values equal to sums of ones of the weighted first level risk values.

In further features, calculating the third level risk values includes setting the third level risk values equal to sums of ones of the weighted second level risk values.

In further features, calculating the overall risk value includes setting the overall risk value equal to a sum of the weighted third level risk values.

In further features, one of the first level risk values is a regulatory compliance risk value, and the one of the first predetermined weighting values associated with the regulatory compliance risk value is greater than all of the other ones of the first predetermined weighting values.

In further features, obtaining the first level risk values includes obtaining at least one of the first level risk values from an electronic drug vendor query.

In further features, obtaining the first level risk values includes storing at least one of the first level risk values in response to user input regarding the at least one of the first level risk values.

In further features, obtaining the first level risk values includes retrieving at least one of the first level risk values from a database.

In further features, the first level risk values are each a value between 0 and 100, and the first predetermined weighting values are values between 0 and 100 percent.

In further features, the second level risk values are each a value between and 100, and the second predetermined weighting values are values between 0 and 100 percent.

In further features, the third level risk values are each a value between 0 and 100, and the third predetermined weighting values are values between 0 and 100 percent.

In further features, the first level risk values include at least 10 different first level risk values associated with the vendor of the drug.

In further features, the second level risk values include at least 5 different second level risk values.

In further features, the third level risk values include 2 different third level risk values.

In a feature, a system for ordering a drug from a vendor of the drug includes: memory including instructions; and one or more processors configured to execute the instructions to: obtain first level risk values associated with the vendor of the drug using a first application programming interface (API), the first level risk values representing real world quantities used to evaluate risk; retrieve first predetermined weight values associated with the first level risk values, respectively, from a SQL database; apply the first predetermined weight values to the first level risk values to obtain weighted first level risk values, respectively; calculate second level risk values associated with the vendor based on the weighted first level risk values; retrieve second predetermined weight values associated with the second level risk values, respectively, from the SQL database; apply the second predetermined weight values to the second level risk values to obtain weighted second level risk values, respectively; calculate third level risk values associated with the vendor based on the weighted second level risk values; retrieve third predetermined weight values associated with the third level risk values, respectively, from the SQL database; apply the third predetermined weight values to the third level risk values to obtain weighted third level risk values, respectively; calculate an overall risk value associated with the vendor based the weighted third level risk values; in response to receipt of user input for the overall risk value for the vendor of the drug, display a visual indicator of the overall risk value of the vendor of the drug on a display; change one of the first, second, and third level risk values and recalculate the overall risk value using the changed one of the first, second, and third level risk values via retrieval using a second API; visually indicate on the display all of the first, second, and third risk values that drop below a first threshold in response to the changing of the one of the first, second, and third level risk values; transmit an electronic alert to an associated device when one of the first, second, and third level risk values exceeds a second threshold; visually change a risk indicator as at least one of the first level, second level, third level, and overall risk values increases; and based on the overall risk value, selectively order a quantity of the drug from the vendor.

In a feature, a method of ordering a drug includes: by one or more processors, using a first application programming interface (API), obtaining first level risk values associated with the drug, the first level risk values representing real world quantities used to evaluate risk; by the one or more processors, retrieving first predetermined weight values associated with the first level risk values, respectively, from a SQL database; by the one or more processors, applying the first predetermined weight values to the first level risk values to obtain weighted first level risk values, respectively; by the one or more processors, calculating second level risk values associated with the drug based on the weighted first level risk values; by the one or more processors, retrieving second predetermined weight values associated with the second level risk values, respectively, from the SQL database; by the one or more processors, applying the second predetermined weight values to the second level risk values to obtain weighted second level risk values, respectively; by the one or more processors, calculating an overall risk value associated with the drug based the weighted second level risk values; by the one or more processors, in response to receipt of user input for the overall risk value for the drug, displaying a visual indicator of the overall risk value for the drug on a display; by the one or more processors, changing one of the first and second level risk values and recalculating the overall risk value using the changed one of the first and second level risk values via retrieval using a second API; by the one or more processors, visually indicating on the display all of the first and second level risk values that drop below a first threshold in response to the changing of the one of the first and second level risk values; by the one or more processors, transmitting an electronic alert to an associated device when one of the first and second risk values levels exceeds a second threshold; and by the one or more processors, visually changing a risk indicator as at least one of the first level, second level, and overall risk values increases; and by the one or more processors, based on the overall risk value for the drug, selectively ordering a quantity of the drug from a vendor of the drug.

In further features, calculating the second level risk values includes setting the second level risk values equal to sums of ones of the weighted first level risk values.

In further features, calculating the overall risk value includes setting the overall risk equal to a sum of the weighted second level risk values.

In further features, obtaining the first level risk values includes obtaining at least one of the first level risk values from an electronic drug vendor query.

In further features, obtaining the first level risk values includes storing at least one of the first level risk values in response to user input regarding the at least one of the first level risk values.

In further features, obtaining the first level risk values includes retrieving at least one of the first level risk values from a database.

In further features, the first level risk values are each a value between 0 and 100, and the first predetermined weighting values are values between 0 and 100 percent.

In further features, the second level risk values are each a value between and 100, and the second predetermined weighting values are values between 0 and 100 percent.

In further features, the first level risk values include at least 10 different first level risk values associated with the drug.

In further features, the second level risk values include 4 different second level risk values.

In a feature, a method of determining a risk associated with a drug and a vendor of that drug includes: by one or more processors, using a first application programming interface (API), obtaining first level risk values associated with the vendor of the drug, the first level risk values associated with the vendor of the drug representing real world quantities used to evaluate risk; by the one or more processors, retrieving first predetermined weight values associated with the first level risk values associated with the vendor, respectively, from a SQL database; by the one or more processors, applying the first predetermined weight values to the first level risk values associated with the vendor to obtain weighted first level risk values associated with the vendor, respectively; by the one or more processors, calculating second level risk values associated with the vendor based on the weighted first level risk values associated with the vendor; by the one or more processors, retrieving second predetermined weight values associated with the second level risk values associated with the vendor, respectively, from the SQL database; by the one or more processors, applying the second predetermined weight values to the second level risk values associated with the vendor to obtain weighted second level risk values associated with the vendor, respectively; by the one or more processors, calculating third level risk values associated with the vendor based on the weighted second level risk values associated with the vendor; by the one or more processors, retrieving third predetermined weight values associated with the third level risk values associated with the vendor, respectively, from the SQL database; by the one or more processors, applying the third predetermined weight values to the third level risk values associated with the vendor to obtain weighted third level risk values associated with the vendor, respectively; by the one or more processors, calculating a first overall risk value associated with the vendor based the weighted third level risk values associated with the vendor; by the one or more processors, using the first API, obtaining first level risk values associated with the drug, the first level risk values associated with the drug representing real world quantities used to evaluate risk; by the one or more processors, retrieving fourth predetermined weight values associated with the first level risk values associated with the drug, respectively, from the SQL database; by the one or more processors, applying the fourth predetermined weight values to the first level risk values associated with the drug to obtain weighted first level risk values associated with the drug, respectively; by the one or more processors, calculating second level risk values associated with the drug based on the weighted first level risk values; by the one or more processors, retrieving fifth predetermined weight values associated with the second level risk values associated with the drug, respectively, from the SQL database; by the one or more processors, applying the fifth predetermined weight values to the second level risk values associated with the drug to obtain weighted second level risk values associated with the drug, respectively; by the one or more processors, calculating a second overall risk value associated with the drug based the weighted second level risk values associated with the drug; by the one or more processors, retrieving sixth and seventh predetermined weight values associated with the first and second overall risk values, respectively, from the SQL database; by the one or more processors, applying the sixth and seventh predetermined weight values to the first and second overall risk values to obtain weighted first and second overall risk values, respectively; by the one or more processors, calculating a third overall risk value associated with the vendor supplying the drug based on the weighted first and second overall risk values; and by the one or more processors, in response to receipt of user input for the third overall risk value, displaying a visual indicator of the third overall risk value on a display; by the one or more processors, changing one of the first, second, and third level risk values and recalculating the third overall risk value using the changed one of the first, second, and third level risk values via retrieval using a second API; by the one or more processors, visually indicating on the display all of the first, second, and third level risk values that drop below a first threshold in response to the changing of the one of the first, second, and third level risk values; by the one or more processors, transmitting an electronic alert to an associated device when one of the first, second, and third level risk values exceeds a second threshold; by the one or more processors, visually changing a risk indicator as at least one of the first level risk values, the second level risk values, the third level risk values, the first overall risk value, the second overall risk value, and the third overall risk value increases; and by the one or more processors, based on the third overall risk value associated with the vendor supplying the drug, selectively ordering a quantity of the drug from the vendor of the drug.

In further features, calculating the third overall risk value includes setting the third overall risk value equal to a sum of the weighted first and second overall risk values.

Further areas of applicability of the present disclosure will become apparent from the detailed description, the claims and the drawings. The detailed description and specific examples are intended for purposes of illustration only and are not intended to limit the scope of the disclosure.

### BRIEF DESCRIPTION OF THE DRAWINGS

FIG. 1 is a block diagram of an example system, according to an example embodiment;

FIG. 2 is a block diagram of an example analysis device that may be deployed within the system of FIG. 1, according to an example embodiment;

FIG. 3 is a block diagram of an example analysis device that may be deployed within the system of FIG. 1, according to an example embodiment;

FIG. 4 is a block diagram of an example user device that may be deployed within the system of FIG. 1, according to an example embodiment;

FIG. 5 is a block diagram of an example participant device that may be deployed within the system of FIG. 1, according to an example embodiment;

FIG. 6 is a display including drug vendor ratings, according to an example embodiment;

FIG. 7 is a display including drug vendor ratings based on an engagement, according to an example embodiment;

FIG. 8 is a display including a rating based on engagements with drug vendors, according to an example embodiment;

FIG. 9 is a block diagram of an example risk elements device that may be deployed with the system of FIG. 1, according to an example embodiment;

FIG. 10 is a block diagram of an example drug data device that may be deployed with the system of FIG. 1, according to an example embodiment;

FIG. 11 is a block diagram of an example risk device that may be deployed with the system of FIG. 1, according to an example embodiment;

FIG. 12 is an example process flow illustrating a method for rating drug vendors, according to an example embodiment;

FIG. 13 an example process flow illustrating a method for rating drug vendors, according to an example embodiment;

FIG. 14 is a block diagram of an example system, according to an example embodiment;

FIG. 15 is a block diagram of an example regulatory device, according to an example embodiment;

FIG. 16 is a block diagram of an example external sources device, according to an example embodiment;

FIG. 17 is a block diagram of an example vendor device, according to an example embodiment;

FIG. 18 is a block diagram of an example purchasing organization device, according to an example embodiment;

FIG. 19 is a block diagram of an example risk element device, according to an example embodiment;

FIG. 20 is a block diagram of an example vendor risk device, according to an example embodiment;

FIG. 21 is a block diagram of an example product risk device, according to an example embodiment;

FIG. 22 is a block diagram of an example engagement risk device, according to an example embodiment;

FIGS. 23A-D are an example risk analysis and evaluation methods performed by a risk analysis and evaluation device, according to an example embodiment;

FIG. 24 is a block diagram of an output device, according to an example embodiment;

FIG. 25 is a block diagram of an example risk views device, according to an example embodiment;

FIG. 26 is a block diagram of an example reports and dashboard device, according to an example embodiment;

FIG. 27 is a block diagram of a notifications device, according to an example embodiment;

FIG. 28 is a block diagram of a machine in the example form of a computer system within which a set of instructions for causing the machine to perform any one or more of the methodologies discussed herein may be executed or stored;

FIG. 29 is a view of a display showing a vendor risk scorecard, according to an example embodiment;

FIG. 30 is a view of a display showing a vendor risk scorecard, according to an example embodiment;

FIG. 31 is a view of a display showing multiple vendor risk scorecards, according to an example embodiment;

FIGS. 32A-D are views of a display showing product data scorecards, according to an example embodiment;

FIG. 33 is a functional block diagram of an example ordering system; and

FIG. 34 is a flowchart depicting an example method of automatically ordering a drug based on risk associated with one of the drug and a vendor of the drug.

### DETAILED DESCRIPTION

Medical costs are ever increasing in the United States. Some medical care facilities and pharmacies have turned to drug sourcing companies to use larger purchasing power to reduce the cost of medicines. A drug sourcing company may contract to supply hundreds, if not thousands, of different drugs. As such, it is challenging for a single person to track and assess risks associated with all of the supplied drugs and the many different vendors and manufacturers of the drugs. This is particularly true for drugs that are off patent and have multiple suppliers.

Example methods and systems for data evaluation and management are described. The performance evaluation may allow a pharmacy drug sales organization, e.g., a group purchasing organization (GPO) and/or a private label distributor, to collect and use data to evaluate risk using methods and systems in a novel manner. Such novel methods may not be available by a person acting with merely mental steps. Moreover, the use of the present systems and methods enables an organization to react quickly to collection and use of data reflecting a change in the underlying factors associated with risk. For example, a regulatory ban against a single manufacturing plant can ripple through the drug supply if that plant supplied an ingredient to one or more drug manufacturers. Additionally, catastrophic weather can damage manufacturing, warehouses, and supply chains. The present systems and methods can capture data that can be used by the methods and systems to account for these disruptions rapidly (e.g., in a matter of minutes or seconds).

In the following description, for purposes of explanation, numerous specific details are set forth in order to provide a thorough understanding of example embodiments. It will be evident, however, to one of ordinary skill in the art that embodiments of the invention may be practiced without these specific details.

In some embodiments, the methods and systems enable drug supply organizations, drug wholesalers, retailers and pharmacies to identify effective and efficient strategies to improve drug supplies by utilizing data to identifying risks in the supply chain. In some embodiments, the methods and systems link the risk to contracting, supply sources, and business models through the use of data. These methods and systems may be used by a group purchasing organization of which drug wholesalers, pharmacies, retail stores having a pharmacy, mail order pharmacy, pharmacy benefit managers, and/or other participants.

In some embodiments, the methods and systems capture historical drug demand data and supplier performance data that are used with analysis to make projections associated with a supplier, a drug type, or both. The methods and systems can also use the captured data to build and test models over various scenarios to improve risk measurement associated with various supply and demand situations for drugs.

The organization using the presently described systems and methods may use such systems and methods for improved functionality with the same or external computer systems, as well as outside of computer systems. By way of example, the methods and systems may be used to assist in negotiating, entering into, and performing certain administrative services related to purchase contracts with drug vendors. The purchase contracts set forth the pricing and other terms and conditions pursuant to which the organization participants will purchase the contracted products, e.g., drugs, from the drug vendors. The organization determines the effective date and term of each purchase contract and the number and type of purchase contracts on which it wishes to enter. The organization may purchase drugs on behalf of participants associated with the organization. In an example, a participant must participate in each contract in the products program. The organization will negotiate prices and terms as well as conditions of, and otherwise perform certain administrative services relating to, the purchase contracts, but will not purchase or take possession of any contracted products or otherwise have the authority to bind participant without participant's prior written permission. The participant will pay the drug supplier directly and be responsible for the goods when ownership transfers to the participant.

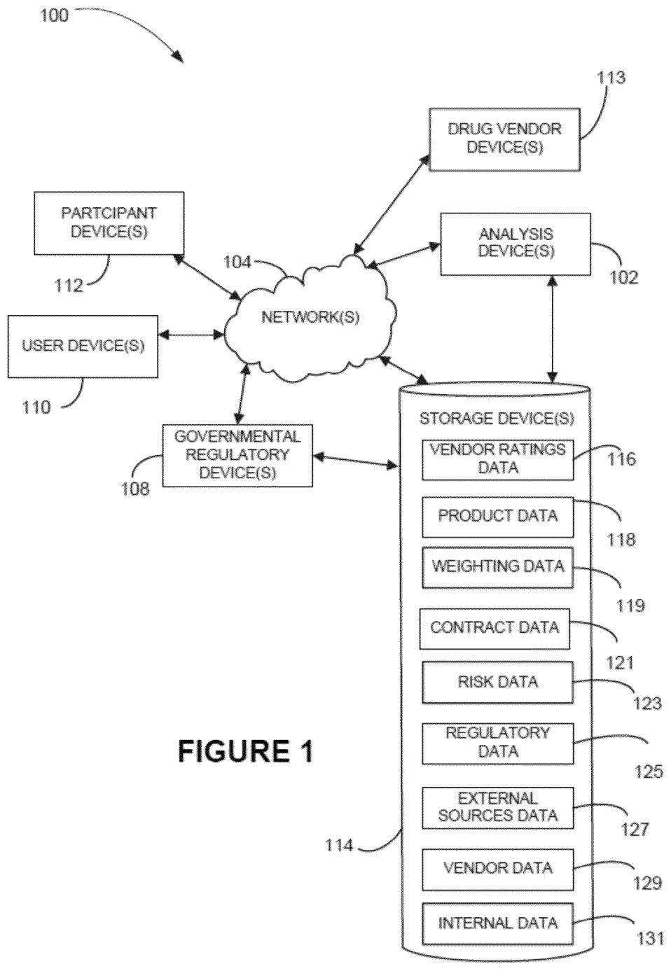

FIG. 1 is a block diagram of an example system 100, according to an example embodiment. The system 100 is an example environment in which data evaluation and management may be performed. The system 100 may include an analysis device 102 in communication with a governmental regulatory device 108, a user device 110, and/or a participant device 112 over a network 104.

The analysis device 102 is a device operated by or at the direction of an entity at least partially responsible for assessing risk in a drug supply, e.g., from a drug vendor. The analysis device 102 may be operated by a purchasing organization, a generic purchasing organization, or the like. In some embodiments, the analysis device 102 may provide server-side functionality to another device. The analysis device 102 may include circuitry, e.g., a memory and a processor associated with the memory.

The network 104 is the means by which the analysis device 102 communicates with the governmental regulatory device 108, the user device 110, and/or the participant device 112. Examples of the network 104 include Mobile Communications (GSM) network, a code division multiple access (CDMA) network, 3rd Generation Partnership Project (3GPP), an Internet Protocol (IP) network, a Wireless Application Protocol (WAP) network, a WiFi network, or an IEEE 802.11 standards network, as well as various combinations thereof. The network 104 may include optical network. The network 104 may be a local area network or a global communication network, such as the Internet. In some embodiments, the network 104 may include a network dedicated to prescription orders, e.g., a prescribing network such as the electronic prescribing network operated by Surescripts of Arlington, Virginia. Other conventional and/or later developed wired and wireless networks may also be used. Any of the devices 102, 108, 110, 112, and 113 may be remote from each other and remote from the storage device 114.

The governmental regulatory device 108 is a device or more than one device operated by or at the direction of an entity at least partially responsible for providing and/or managing governmental regulatory programs. For example, the governmental regulatory device 108 may be operated by or at the direction of US Department of Health and Human Service or the Food and Drug Administration (FDA). In general, the FDA is responsible for protecting and promoting public health through the regulation and supervision of prescription drugs, over-the-counter pharmaceutical drugs (medications), vaccines, biopharmaceuticals, and veterinary products. Other national or transnational drug oversight organizations can operate a governmental regulatory device 108, e.g., Health Canada, National Health Service of the UK, World Health Organization, Secretariat of Health of Mexico, and corresponding organizations around the world. Data relating to any excluded drug manufacturer or excluded drug can be retrieved or downloaded using the governmental regulatory device 108.

In an example embodiment, the Office of the Inspector General (OIG) may issue an exclusion order that bans a particular drug from a particular manufacturer from importation into the United States. Exclusion orders may also be issued to component suppliers who supply components used by a drug manufacturer to manufacture a drug. Exclusion orders may identify a particular drug, a generic drug, a component of a drug, a manufacturer of the drug, the manufacturer or wholesaler of a component of the drug, or other organizations involved in the production of the drug. Similar exclusion orders may be issued by other government organizations. For example, each manufacturer's name may be entered into a search field in a database accessible through the governmental regulatory device 108 and the results downloaded and stored in the current storage device 114. The availability and use of such data enables the calculated risk evaluation associated with any given drug vendor to also include its past performance history as a component.

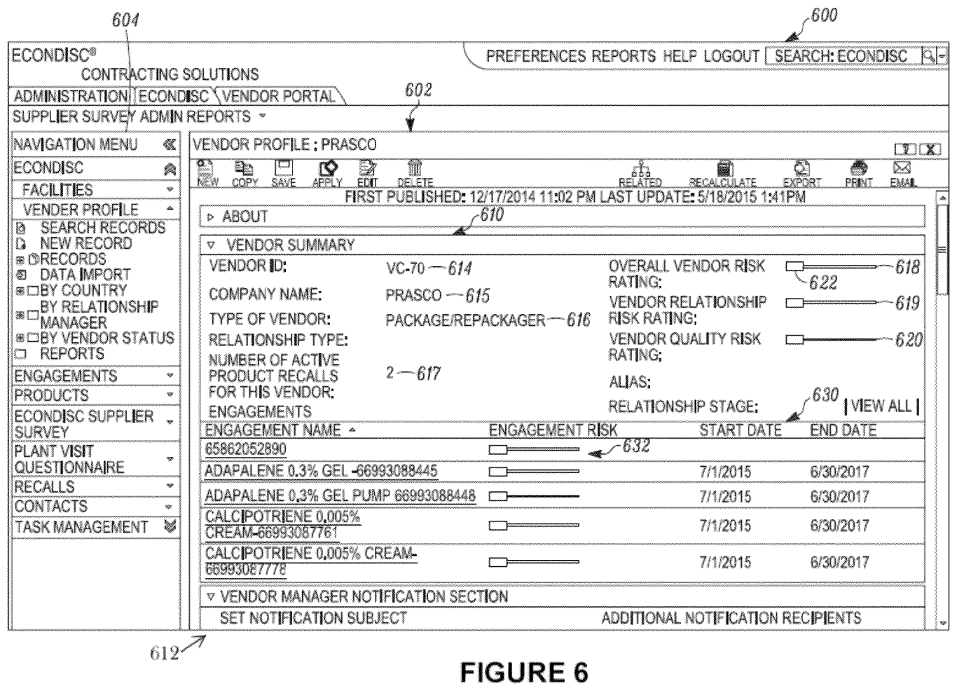

The user device 110 is used by a device operator. The user device 110 may be a stand-alone device that solely provides at least some of the functionality to enable data evaluation and management (e.g., to analyze drug vendor performance or risk of supply of any or all drugs from the vendor). In some embodiments, the user device 110 is used in conjunction with the analysis device 102 such that the analysis device 102 provides server-side functionality and the user device 110 provides client-side functionality. For example, the user device 110 may be used by a representative associated with the analysis device 102 to present information associated with vendor performance and risk relative to its ability to provide a drug. An example of risk that can be evaluated in the analysis device 102 can be risk or predicted risk of drug supply interruption based at least in part on predicted drug needs. The predicted drug needs may be predicted by using claims data from a health plan. The user device 110 may be operated by the purchasing organization, a group purchasing organization or the like, as well as by other participants in the evaluation of a drug supply. The user device 110 may output reports or results relating to risk, which can be determined as described herein. Examples of such reports are shown in FIGS. 6-8 below.

The participant device 112 is a device operated by an entity at least partially responsible for the management of a drug and/or medical benefit program. The participant device 112 may be associated with a participant organization, for example, a purchasing organization, a drug purchasing organization, a group purchasing organization or the like. In general, a group purchasing organization and/or a private label distributor may negotiate and purchase generic drugs on behalf of pharmacies and other drug sellers. While the entity operating the participant device 112 may be a purchasing organization (e.g., a group purchasing organization), other entities may operate the participant device 112 either on behalf of themselves, a pharmacy benefits manager (PBM), a pharmacy, a mail order pharmacy or another entity. In some embodiments, the participant that provides the drug may also provide one or more than one additional benefits including a health benefit, a dental benefit, a vision benefit, a wellness benefit, a radiology benefit, a pet care benefit, an insurance benefit, a long term care benefit, a nursing home benefit, and the like. The participant device 112 may also provide demand numbers for particular drugs to the other devices 102, 104, 108, 110, 114.

A PBM may have members (or a person on behalf of the member) that attempts to obtain a prescription drug at a retail pharmacy location where the member can obtain drugs in a physical store from a pharmacist or pharmacist technician, or in some instances through mail order drug delivery from a mail order pharmacy location. The prescription drug may otherwise be received through a different delivery channel such as a prescription drug kiosk station.

The member may have a co-pay for the prescription drug that reflects an amount of money that the member is responsible to pay the pharmacy for the prescription drug. The money paid by the member to the pharmacy may come from the personal funds of the member, a health savings account (HSA) of the member or the member's family, a health reimbursement arrangement (HRA) of the member or the member's family, a flexible spending accounts (FSA) of the member or the member's family, or the like. An employer of the member and/or a prescription/health/medical benefit plan may directly or indirectly fund or reimburse the member or an account of the member for the co-pay.

The amount of the co-pay paid by the member may vary by the benefit plan of the client with the PBM. The member's co-pay may be based on be a flat co-pay (e.g., $10), co-insurance (e.g., 10%), and/or a deductible (e.g., for first $500 of annual prescription drug spend) for certain prescription drugs, certain types of prescription drugs, and/or all prescription drugs.

In certain instances, the member may not pay the co-pay or may only pay for a portion of a co-pay for a prescription drug. For example, if the usual and customary cost for a generic version of a prescription drug is $4, and the member's flat co-pay is $20 for the prescription drug, the member may only pay $4 to receive the prescription drug. In another example involving a worker's compensation claim, no co-pay may be due by the member for the prescription drug.

In conjunction with receiving the co-pay (if any) from the member and dispensing the prescription drug to the member, the pharmacy submits a claim to the PBM for the prescription drug. The PBM may perform certain adjudication operations including verifying the eligibility of the member, reviewing the formulary to determine appropriate co-pay, coinsurance, and deductible for the prescription drug, and performing a drug utilization review (DUR) on the member. The PBM then provides a response to the pharmacy following performance of the aforementioned operations. As part of the adjudication, the client (or the PBM on behalf of the client) ultimately reimburses the pharmacy for filling the prescription drug when the prescription drug was successfully adjudicated. The aforementioned adjudication operations generally occur before the co-pay is received and the prescription drug dispensed. However, the operations may occur simultaneously, substantially simultaneously, or in a different order. In addition, more or less adjudication operations may be performed as part of the adjudication process. Any of the data associated (e.g., claims data and the like) with any drug benefit or adjudication provided by the PBM can be used in the measures for determining a rating for the drug vendor or drug supply, e.g., risk or evaluation, by the GPO. The PBM can also determine and communicate drug needs using prescription information to other device in system 100, e.g., to the storage device 114 or the GPO and its related devices to evaluate the possible drug supply disruption or to determine risk to drug supply.

In some example embodiments, the PBM creates prescription drug event records for each drug benefit claim. Such drug event records may be used to create a summary record in view of other data, e.g., public data or market information. The summary record is created using this data to create a prescription drug event (PDE) data. In an example, the GPO creates the PDE data. PDE data can reflect the real-time demand or demand trend for a particular drug supplied by a drug vendor. The PDE data is not the same as individual drug claim transactions, e.g., prescription drug cost and payment data. The PDE data may be used to determine demand for a drug at any given time, e.g., monthly, yearly, seasonally, likelihood of steep changes in demand and the like. The PDE data can include data relating to a generic drug. In some example embodiments, the PDE relates to nongeneric drugs or components of drugs that are tracked by the GPO or in the database associated with the PBM.

An example of a PDE may include a warning letter to a drug manufacturer. A manufacturer of finished dose receives a warning letter for one of their manufacturing plants by a governmental agency, e.g., the FDA. The remediation efforts implemented by the manufacturer will slow down production and may also result in a large recall. The product specific manufacturing location within the database and the product risk algorithm allows us to identify the products impacted and the appropriate risk mitigation strategies to be implemented very quickly, usually within a few hours of the warning letter being issued by the governmental agency.

Another example of a PDE may include a raw material manufacturer being placed under an import ban by a governmental agency, e.g., FDA. This impacts production for multiple drug manufacturers sourcing from the impacted raw material supplier. The raw material information within the risk database (e.g., data storage 114) allows us to identify quickly manufacturers that are impacted, manufacturers that are not and then use the product and vendor risk to implement risk mitigation strategies such as moving the drug supply to non-impacted manufacturers ahead of other drug purchasers. This will minimize disruption of the drug supply for the participants.

Another example of a PDE may include a slow down or backorder status of a manufacturer. A manufacturer is identified as having large backorders, either in number of orders or quantity of drug doses. These backorders are not being resolved in a timely manner by the manufacturer. That is, the backorders last over a time threshold. The present devices, systems and methods for evaluating drug supply can track backorder risk to apply appropriate risk mitigation strategies and minimize drug supply disruption.

In an example embodiment, the GPO may access the drug claims data that may be part of a pharmacy database or a drug claims adjudicator database. The claims data may be used to derive the PDE data. The PDE data may be macro level data that is derived from micro-level data, e.g., individual drug claims data.

The GPO, either by operation of its own pharmacy or by contractual agreements, has an interest in ensuring a proper supply of drugs to the members of pharmacy benefits plan or to the patients that fill prescriptions at a pharmacy that is part of the GPO. Such a pharmacy is an example of a participant in the GPO. Other participants in the GPO may be drug wholesalers or other drug suppliers. The group purchasing organization and/or a private label distributor may assess the risk of a supply of a drug from a vendor to participants. Examples of risks may include the risk associated with the drug manufacturer, risk of a component supply to the vendor, and risk of a particular drug. In general, a risk may be the likelihood that a drug supply will be disrupted (e.g., to the extent that a patient may not receive a prescribed drug, a price of a drug may spike out of an acceptable range, or the like). Another risk may be safety of the drug. For example, a patient not being able to take the drug due to safety of the drug being compromised due to violations of a contract obligation, current good manufacturing processes, governmental regulations, or other safety violations by the manufacturer. Risk may include the risk of a patient not receiving the drug due to non-availability, patient being not able to afford the drug due to prices increasing out of an acceptable range. Risk, in example embodiments described herein, relate to the risk of unavailability of providing certain drugs that may otherwise be prescribed to a patient. As such, the risk may be in terms of potential impact on patient health. Other forms of risk may be evaluated as described herein.

Some of the operations of the group purchasing organization and/or a private label distributor that operates the participant device 112 may include the following. The weighting data for various risk factors may be stored or altered in the participant device 112. The participant device 112 may also store data relating to contractual relationships between drug supplying organizations and the group purchasing organization and/or private label distributor. The GPO and/or private label distributor may be part of the PBM and supplies reliability and risk information to the PBM or to others, e.g., participants and participant devices 112. The GPO and/or private label distributor may quantify reliability and risk information related to generic drugs or drug components.

A drug vendor device 113 may be a machine associated with the manufacturer or seller of a pharmaceutical or other regulated drug. The machine may include circuitry, e.g., a memory and a processor associated with the memory, to provide data required by governmental regulators, government regulation devices, or data required by the GPO and its related devices. The drug vendor device 113 may be a device for a wholesaler of a drug or a distributor for a drug. The drug vendor device 113 may report data regarding manufacture of drugs or components of drugs. Such data may include type of drug being supplied by the vendor, suppliers of raw ingredients used to manufacture the drug, suppliers of the active pharmaceutical ingredient, status of back orders, among other. The drug vendor device 113 may further report data representing the status of the manufacturers' infrastructure including, but not limited to, vertical integration data, plant status, plant shut down, plant upgrades, future maintenance plans and the like. Other types of data that may be reported by the drug vendor device 113 include warnings from governmental regulators and import bans imposed on the vendor. The vendor may choose to provide the aforementioned data to be consider as a possible vendor, may be obligated to provide the aforementioned data pursuant to contractual obligations, or may otherwise provide the aforementioned data.

The analysis device 102 may be in a client-server relationship with the devices 108, 110, 112, 113 a peer-to-peer relationship with the devices 108, 110, 112, 113 or in a different type of relationship with the devices 108, 110, 112, 113.

The analysis device 102, the governmental regulatory device 108, and/or other devices 110, 112, 113 may be in communication directly (e.g., through local storage) and/or through the network 104 (e.g., in a cloud configuration or software as a service) with a storage device 114. The devices 102, 108, 110, 112, 113 may share a storage device, have separate storage devices, or share one or more storage devices and also have separate storage devices. In an example, some devices 102, 108, 110, 112, 113 have different levels of access to data in the storage device 114.

The storage device 114 may be deployed on one or more that one of the devices 102, 108, 110, 112, partially on more than one of the devices 102, 108, 110, 112, or may otherwise be deployed. The deployment may occur on local storage, remote storage, removable storage, and/or a different type of storage associated with the devices 102, 108, 110, 112. Additionally, while a single storage device is depicted, multiple storage devices may be implemented. In the case of multiple storage devices, the different storage devices may be deployed on different systems, including the devices 102, 108, 110, 112, and/or a third-party device or network.

The storage device 114 may include: a non-transitory storage (e.g., memory, hard disk, CD-ROM, etc.) in communication with the devices 102, 108, 110, 112, either directly and/or over the network 104. The non-transitory storage may store any data as described herein.

The storage device 114 may store vendor ratings data 116, product data 118, and the like. The storage device 114 may also store different types of data, e.g., weighting data 119 and contract data 121. The storage device 114 may further store risk data 123, regulatory data 125, external sources of data 127, vendor data 129 and/or other internal data 131. The regulatory data 125 reflects data from or associated with regulatory bodies, e.g., governmental agencies that track and regulate drug supply and drug vendors.

The external sources data 127 reflects data relating to political, socio-economic, economic, weather, and the like. These may relate to a drug supply being evaluated by the purchasing organization.

The vendor data 129 reflects data that ranks the vendor in certain categories. The vendor data 129 may be derived from other data, input by a person reflecting knowledge relating to a vendor, or otherwise. In some embodiments, the vendor data 129 reflects interpretation of various aspects of a vendor, e.g., the technical capabilities of a vendor, compliance data, weather data, supplier to the vendor data, regulatory data, and the like. Other data that can be part of the vendor data 129 may include calculated engagement risk, drug delivery timeliness, back orders, infrastructure, management data, and financial health. In calculating the derived data, the components used for calculation may also be stored as part of the vendor data 129.

The vendor data 129 may also include data that reflects data for a vendor or from the vendor. The vendor data 129 may include the vendor identifying information and drug identifying information for all drugs being supplied or manufactured by each vendor. The vendor need not be under any agreement to supply the drug to the participant(s) of the group purchasing organization, or any organization associated with the devices described with reference to FIG. 1. However, such data may be used to evaluate the vendor performance and calculate a risk for other drugs from the vendor. In general, a vendor may be a manufacturing entity (e.g., that physically creates the drug), a packaging entity (e.g., that packages the drug created by another) or a wholesaling entity (e.g., that sells the drug created by another at wholesale to an entity such as a pharmacy for further sale to a patient). Pharmaceutical wholesalers purchase and distribute large volumes of prescription drugs and over the counter drugs from manufacturers of brand and generic drugs by consolidating demand from their clients, e.g., participants. This allows the wholesaler to leverage volume to lower the drug acquisition cost for their clients. Their clients include retail pharmacies, hospitals, physicians and alternate site customers. The wholesaler may also provide additional services to their clients such as logistics and inventory management and various other administrative services. The organization using the present system and methods may act as a wholesaler to the participants in the purchasing organization.

The product data 118 reflects data associated with a drug product that is or may be part of the purchasing made by the purchasing organization (PO) or generic purchasing organization (GPO). The product data can include the drug name, other identifying information, the active ingredients, the inactive ingredients, the suppliers of the ingredients, the manufacturer of the drug.

The organization internal data 131 may include contractual obligations and the drug vendors' abilities and historical performance in meeting the contractual obligations.

The weighting data 119 reflects a weight to any factor or weighting component being used to calculate risk in the drug supply. Weighting data 119 may be used to define how certain types of data are used in evaluation drug supply. For example, some data may be more heavily weighted than others and the weights in the weighting data 119 may be changed in the analysis device 102.

The contract data 121 may be used to track criteria relating to contractual obligations of a drug vendor. If a risk factor is neutral it may be assigned a neutral weighting, e.g., one. If the risk factor is to be a greater factor in calculating a risk, then it is assigned a heaver weight than other factors in the calculation, e.g., greater than one. If the risk factor is to be a lesser factor in calculating a risk, then it is assigned a lighter weight than other factors in the calculation, e.g., less than one. Other weighting schemes may also be used.

At least one of the analysis device 102, the user device 110, and the participant device 112 operate to evaluate drug vendor performance. Such device or devices may store, weigh, process and display various risk elements and risk assessments. The data and risk elements may be weighted so that data or a risk element that has a greater effect on risk or drug supply is given greater weight when processing risk assessments. The devices 110, 112 may store or process data relating to a drug vendor. Such data may include any one or more of financial health of a drug vendor, contract history with a drug vendor, current contractual obligations of the drug vendor, fee payments, shelf stock adjustments, and the like. These devices 102, 110, and 112 may use any of the data stored in the storage device 114 or input into the devices to generate risk assessments. The risk assessments can be used to manage relationships with drug vendors to assist in ensuring a supply of a drug at an acceptable price. In an example embodiment, the management of the relationship may include setting prices for drugs, securing contractual guarantees, ensuring priority of drug supply, or the like.

While the system 100 in FIG. 1 is shown to include single devices 102, 108, 110, 112, 113 multiple devices may be used. The devices 102, 108, 110, 112, 113 may be the same type of device or may be different device types. When multiple devices are present, the multiple devices may be of the same device type or may be a different device type. Moreover, system 100 shows a single network 104; however, multiple networks can be used. The multiple networks may communicate in series with each other to link the devices 102, 108, 110, 112, 113 or in parallel to link the devices 102, 108, 110, 112, 113. Each of the devices 102, 108, 112, 113 either alone or connected together in ant combination, can operate as a dedicated device specifically programmed to perform a step, a method or any function described herein.

In some embodiments, at least some of the functionality of the analysis device 102 may be included in the user device 110 and/or the participant device 112. The user device 110 or the participant device 112 may communicate with the storage device through the network 104 or through the analysis device 102.

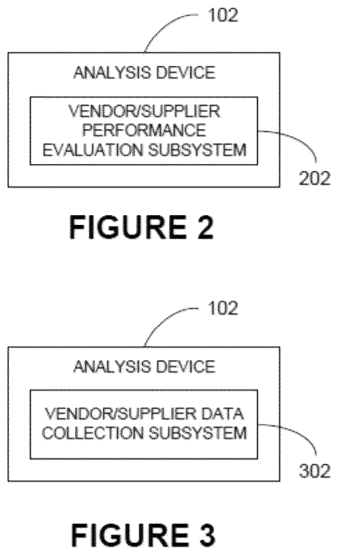

FIG. 2 illustrates the analysis device 102, according to an example embodiment. The analysis device 102 may be deployed in the system 100, or may otherwise be used. The analysis device 102 may include a drug vendor performance evaluation subsystem 202 to evaluate the performance or risk of the drug vendor or drug manufacturer. The drug vendor performance evaluation subsystem 202 may include circuitry that executes a program to access instructions and data stored in the storage device 114 to evaluate drug supply interruption or risks to drug supply that is predicted to be needed by the participants with the GPO.

The vendor performance evaluation subsystem 202 is capable of performing ratings calculations relating to one or more of: a particular drug from a single vendor, a particular drug from multiple vendors, or a vendor as a whole. In some embodiments, such capability is enabled by implementing rules (e.g., stored in a memory operably associated with the subsystem 202) on processing circuitry (a dedicated machine when loaded with the rules). The rules executed by the subsystem 202 may include weightings that give more weight to certain inputs relative to other inputs when calculating the vendor performance and risk.

In some embodiments, the some data associated with a vendor may not be available. In such a case, the subsystem 202 may use an average of data from like vendors to create a value for of the missing data in the calculation of performance or risk.

FIG. 3 illustrates the analysis device 102, according to an example embodiment. The analysis device 102 may be deployed in the system 100, or may otherwise be used. The analysis device 102 may include a drug vendor/supplier data collection subsystem 302 to acquire the data on which the performance or risk of the drug vendor or drug manufacturer can be based. The collection subsystem 302 is capable of performing data retrieval and collection relating to one or more of: a particular drug from a single vendor, a particular drug from multiple vendors, or a vendor as a whole. In some embodiments, such capability is enabled by implementing rules (e.g., stored in a memory operably associated with the subsystem 302) on processing circuitry (a dedicated machine when loaded with the rules). In some embodiments, the some data associated with a vendor may not be available. In such a case, the subsystem 302 may use an average of data from like vendors to create a value for of the missing data in the calculation of performance or risk. The subsystem 302 may further use historical and statistical analysis to fill in missing data. The subsystem 302 may further highlight the missing data to the purchasing organization.

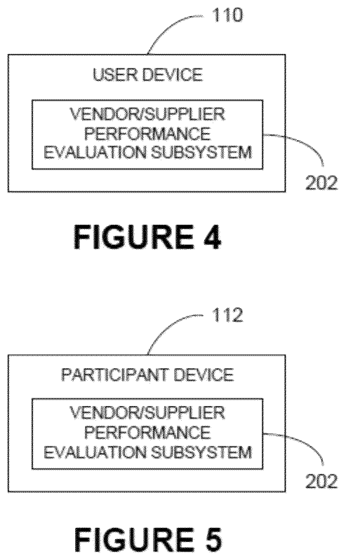

FIG. 4 illustrates the user device 110, according to an example embodiment. The user device 110 may be deployed in the system 100, or may otherwise be used. The user device 110 may include a drug vendor performance evaluation subsystem 202 to evaluate the performance or risk of the drug vendor or drug manufacturer.

FIG. 5 illustrates the participant device 112, according to an example embodiment. The participant device 112 may be deployed in the system 100, or may otherwise be used. The participant device 112 may include a drug vendor performance evaluation subsystem 202 to evaluate the performance or risk of the drug vendor or drug manufacturer.

FIG. 6 illustrates a display 600 that may be generated by the analysis device 102 or other devices associated with the GPO. The display 600 shows a vendor profile 602 of a risk assessment relating to a drug vendor, according to an example embodiment. A vendor profile may show data relating to a vendor and risks that have been calculated related to the vendor. The display 600 may show information or portions of information that can navigated between using selections on the display 600, e.g., using in input/output of the associated device. A navigation menu 604 can provide an efficient way to change the information on the display 600.

The vendor profile tab 603 is selected in the navigation page to show the vendor profile 602 in the display. The vendor profile 602 may store information relating to a vendor, e.g., a drug manufacturer, a drug wholesaler, a drug repackager, or the line involved in the production or distribution of a drug, e.g., a generic drug. The vendor profile 602 includes a vendor summary 610 and a vendor manager notification section 612. The vendor summary 610 shows vendor identifying information, e.g., a vendor ID 614, a vendor name 615, a vendor type 616, a number of active recalls 617, an overall vendor risk rating 618, a vendor relationship risk rating 619, a vendor quality risk rating 620. The vendor summary 610 can also show any alias names of the vendor, e.g., a doing business as and the like, and a relationship stage information. The vendor type 616, here shown as packager/repackager, may be a risk factor used in calculating various risks associated with the vendor. The number of active recalls 617, here shown as “2”, may be a risk factor used in calculating various risks associated with the vendor. The overall vendor risk rating 618 is calculated from various risk factors, as described herein, and displayed as indicia 622 along a spectrum from worse to good. In additional to the location of the indicia (e.g., on a graph), the color of the indicia 622 may also indicate the overall risk associated with the vendor. The overall vendor risk rating may be calculated from other calculated risks, e.g., the relationship risk 619 and the vendor quality risk rating 620, which can be weighted relative to each other to properly calculate the overall risk. In the illustrated example, the overall vendor risk rating 618 is shown with its indicia at its highest level (here, leftmost) and in a green color. If the risk was highest, then the indicia would be at the lowest level (here, rightmost) and in a red color. The line graph displaying risk is shown with multiple levels, here, five. Each level may also have its own color associated with it. The indicia colors may be green, blue, yellow, orange and red to show varying risks from low to high. Other colors could be used for any or each other levels of risk displayed by the indicia 622. Similar indicia may be used for any of the calculated risks described herein, e.g., the vendor relationship risk 619, the vendor quality risk 620, or the like.

The navigation menu 604 shows multiple navigation links including, but not limited to, facilities, vendor profile, engagements, products, supplier survey, plant visit questionnaire, recalls, and contacts. The navigational links when selected may allow the device to change views on the display, e.g., navigational pages on the display, being shown on the display 600, e.g., of a device as described herein. The facilities portion of the display 600 may display risks related to a facility where a drug is manufactured and may allow the input of data related to the facility. The vendor profile page may display risks related to the vendor and may allow input of data related to the vendor. The engagement page may display risks related to the vendor engagements and may allow input of data related to the engagements. Such engagements may include drug supply agreements, contractual relationships, and the like. The products page may display risks related to the particular products or classes of products and may allow input of data related to the products. The supplier survey page may display survey questions to be answered by a supplier and may allow input of data related to the supplier. The plant visit page may display generated survey questions to be answered by a visitor or inspector of a plant associated with either a drug product or a supplier and may allow input of data related to the plant. The plant visit page may display risks related to the vendor and may allow input of data related to the vendor. The recalls page may display recall or government intervention questions to be answered and may allow input of data related to the recall or government interventions for any of a drug, a supplier and an ingredient of a drug. A contacts page may display and allow the updating of contacts associated with any drug or any supplier. Any of the data inputted through the pages may be stored in the storage device, e.g., storage device 114 (FIG. 1), and used to calculate risk, either as a weighted risk factor or a non-weighted risk factor.

The vendor summary 610 may also display the engagements 630 between the vendor and risk assessor. The engagements 630 can be displayed by name with each engagement having its own engagement risk 632 associated with the engagement. Indicia can show the calculated level of risk for any particular engagement. The engagement risk 632 may take into account the past history of the vendor, the source of ingredients for making the drug, whether the vendor owns the manufacturing plant (of either the drug or the ingredients to manufacture the drug), the current governmental regulatory status, global supply of the drug or its ingredients, and the like.

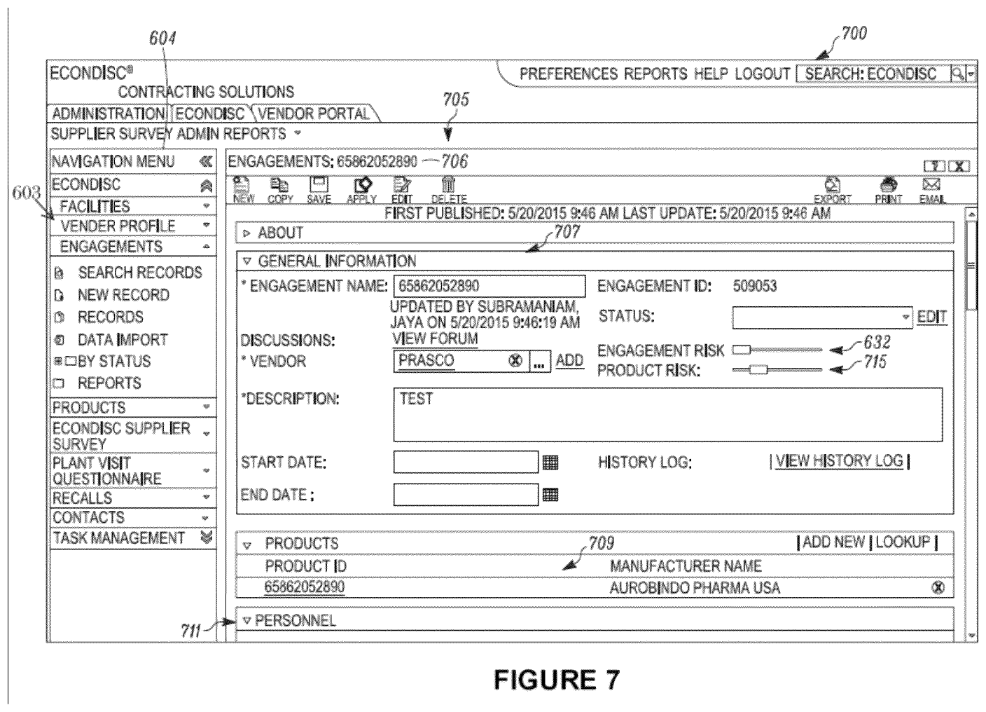

FIG. 7 illustrates a display 700 that may be generated by the analysis device 102 or other devices associated with the GPO. The display 700 can be a view that is a modified view of the information on the display 600. The display 700 shows the navigation menu 604 with engagements selected so that the associated circuitry generates the information on the display 700. The display 700 further shows an engagement section 705 that shows data relating to a particular selected engagement. The engagement section shows the name of the engagement 706, a general information section 707, a products section 709, and a personnel section 711. The general information section 707 gives the name, allows for researching past discussions, names the vendor, allows for display and entry of a description of the engagement, and displays risk associated with the engagement 632 and a product risk 715. The product risk 715 relates to the risk associated with the product or products that is subject to the engagement selected for generating information on the display 700. Here, the product risk 715 is one level below the least risk. The indicia of the product risk 715 is here shown as blue and moved to a lower location, i.e., one spot to the right. The products section 709 shows the products that are subject to the engagement along with the manufacturer of the drug. As shown in FIG. 7, the manufacturer of the drug may be different than the vendor. That is, the vendor may be a wholesaler or repackager who receives the drug and packages same to supply to various markets, which may be oversaw by the PBM or other drug supplier.

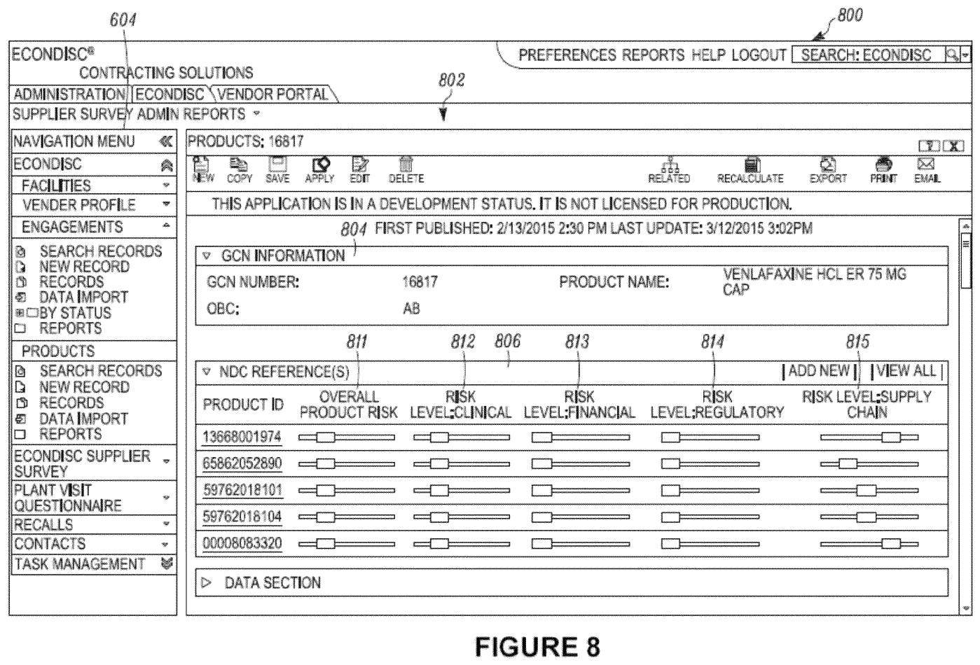

FIG. 8 illustrates a display 800 that may be generated by the analysis device 102 or other devices associated with the GPO. The display 700 may show the navigation menu 604 with the products displayed.

The display 800 further shows a products section 802 that displays data relating to a particular product, here shown as product 16817. A GCN section 804 shows the generic code number (GCN), the product name, and the Orange Book Code (OBC). The OBC identifies the therapeutic equivalency ratings assigned to each approved prescription product according to the FDA's Approved Drug Products with Therapeutic Equivalence Evaluations. An NDC (national drug code) section 806 shows the various products that are available for a product under the same GCN. Here the various risks associated with each available equivalent drug are shown in NDC section 806. The NDC section 806 shows the overall product risk 811, the clinical risk 812, the financial risk 813, the regulatory risk 814, and the supply chain risk 815. The overall product risk 811 is the same for each of the shown five drugs, specifically, a level down from least risk out of the five risk levels shown. The clinical risk 812 is the same for each of the shown five drugs, specifically, a level down from least risk out of the five risk levels shown. The financial risk 813 is the same for each of the shown five drugs, specifically, a least risk (shown as leftmost position in the line graph) of the five risk levels shown. The regulatory risk 814 is the same for each of the shown five drugs, specifically, a least risk (shown as leftmost position in the line graph) of the five risk levels shown. The supply chain risk 815 is the different amongst the five drugs shown. This is due to underlying factors used in calculating the risk for each drug, e.g., the access to underlying ingredients may differ, the location of manufacturing may differ, the status of the country of origin may be different, the manufacturing facilities may differ, etc. The first drug and the last drug shown in FIG. 8 both have a supply chain risk that is second from the lowest (second from right in the five level line graph) and can show the indicators in orange. The third drug and the fourth drug both have a supply chain risk that is intermediate and is shown in the third position in the five level line graph) and can show the indicators in yellow. The second drug has a supply chain risk that is second from the best (second from left in the five level line graph of FIG. 8) and can show the indicators in blue. As shown, none of the drugs has a best (highest or leftmost in FIG. 8) risk or a worst (least or rightmost in FIG. 8) risk in its supply chain risk. In an example embodiment, the overall product risk 811 is calculated from the clinical risk 812, the financial risk 813, the regulatory risk 814, and the supply chain risk 815. Each of the risks 812-815 may be weighted while calculating the overall product risk.

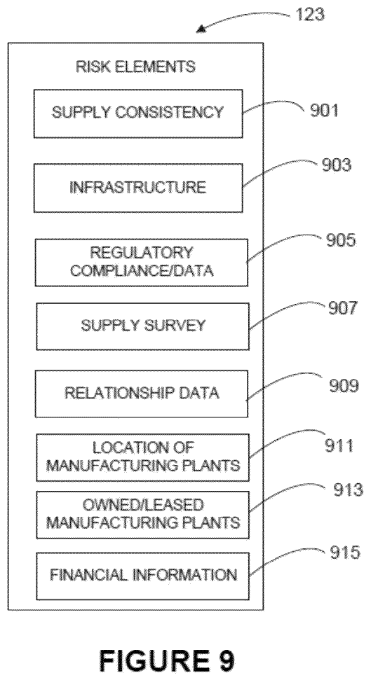

FIG. 9 shows a data storage device 123 that can receive, store, and update risk elements that can be used in the drug supply risk assessments described herein. The device 123 can be a machine, e.g., an electronic machine or a computing system. The device 123 can supply risk elements to the analysis device 102 or user device 110 to determine risk computations using risk elements and evaluate drug supply. The supply consistency 901 includes data related to the meeting of contractual demands for a drug per the terms of the contract. The supply consistency 901 may include data regarding supplies of a drug already manufactured and available for delivery, e.g., drug supply stored in a warehouse and available for shipment. The supply consistency 901 can be derived from tracking the timeliness of a drug delivery per contractual terms by the PBM or the health plan. The supply consistency 901 may also include data reported by the drug supplier, e.g., a vendor, wholesaler and/or the manufacturer. In an example embodiment, the supply consistency 901 can be reported to the data storage device 123 automatically from the data generated by shipping and receiving systems, which may be associated with participants, such as insurers, pharmacies, a PBM, and other participants.

Infrastructure 903 includes information about the drug supplier's physical and organizational structures and facilities (e.g., buildings, plants, production equipment, and power supplies) needed for the manufacture, packaging, repackaging, delivery of a drug to a location of the PBM or health plan's choosing. The infrastructure 903 may include, but is not limited to, manufacturing plants (e.g., capabilities, capacity and number), technology (amount and type), redundancies available on a per drug and a per vendor basis, disaster relief plans and the like.

Regulatory compliance 905 includes information about the vendor meeting governmental regulations and laws or trade group requirements. The regulatory compliance is not limited to a single government and may take into account compliance with governmental regulations in multiple jurisdictions and around the world. For example, the Federal Drug Administration (FDA) or the Office of Inspector General (OIG) may issue a warning to the vendor or ban products from the vendor or from a specific manufacturing plant. In an example embodiment, the FDA may inspect a product or location and issue a warning letter or an import ban that immediately cuts off the drug supply almost immediately. The regulatory compliance 905 may also track recalls, which may cuts off or reduces the supply of a drug within the U.S. market or global market significantly and quickly. In an example embodiment, the regulatory compliance may be assigned the greatest weight in calculating risk.

Supply survey 907 may include data that a drug vendor must input into the system 100 for storage in the storage device 114 using participant device 112, in an example or reply to the risk assessment organization for inclusion in risk assessment. The supply survey 907 may include data regarding timeliness of drug delivery, financial health of vendor, and other data relating to the vendor. The supply survey 907 includes queries that must be answered within certain time periods, e.g., monthly, quarterly, annually, etc., e.g., through the device 102-113 of the system 100 (FIG. 1). The supply survey 907 can be a listing of queries generated by any of one or more than one of the analysis device 102, the user device 110, and the participant device 112. The drug vendor may interact and input responses by using the drug vendor device 113.

Relationship data 909 may include data that relates to relationships between a drug supplier and the organization. The relationship data may be based on pricing strategy, contract obligations, and other relationship data. The pricing strategy may be based on incentives, contractual enhancements, pricing compliance and/or pricing terms. The contractual obligations may be based on pharmaceutical product development fee or administration fee payments, including timelines and timeliness, failure to supply adherence, and completion and accuracy of supplier surveys, which can be sent by the purchasing organization. Additional relationship data 909 may include supplier senior management engagement with the purchasing organization, national account manager engagement with the purchasing organization, and transparency in its interactions with the purchasing organization.

Manufacturing plant location 911 may include data relating to the location of any manufacturing plant of a drug vendor, repackager or supplier of ingredients used to manufacture the drug. By storing the locations of any plant in the manufacturing plant location 911, the system 100 can update risks based on any event occurring at any plant. Events that may affect the manufacture of a drug at any plant location include people centered events, e.g., strikes, revolution, wars, work slow-downs, civil unrest, and the like. Other events include weather related events, e.g., monsoons, flooding, high winds, mudslides, tornados, and the like. In some embodiments, the manufacturing plants are tracked on an individual basis and/or a drug being manufactured basis and grouped by company and location. While some of these vents may be received from the manufacturer directly, e.g., through the use of data entry through an API at the user device, some of the data may be input into the present system through other sources, e.g., governmental sources, news sources, electronic data sources.

Manufacturing plant(s) information 913 may include and store data regarding ownership or leasing of the drug manufacturing plants. The information 913 is used to determine risk.

Financial information 915 stores data relating to the finances of any drug vendor, repackager or supplier. The financial health data 915 may be used in determining risk.

It will be recognized that any of the data and information can be used in the calculation of a risk or evaluation of drug supply. The data and information in device 123 may be weighted before risk calculations to reflect a more true effect on the risk involved.

The data elements (e.g., information) in the device 123 may be input into the system 100 from an operator of a device, a person using the device at a vendor, or through an API. The data is input the system 100 can is translated into a value. Such a value represents a real world quality that is used to evaluate a risk to a drug supply. These values can then be weighted and used in the calculations associated with determining risk to a drug supply.

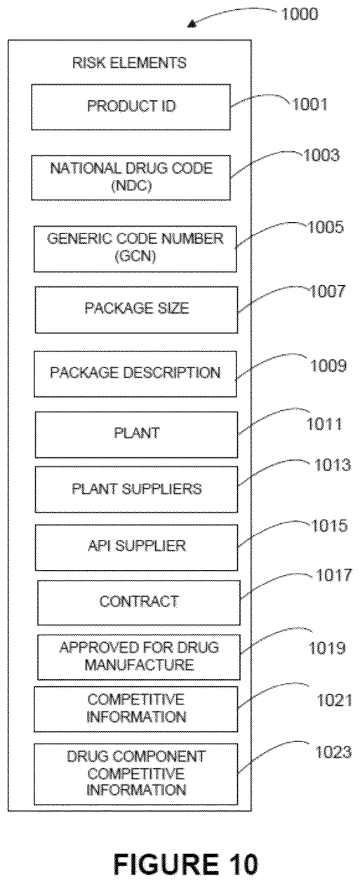

FIG. 10 shows a risk element data storage device 1000 that can receive, store, and update risk elements that can be used in the drug supply risk assessments described herein. The device 1000 can be a machine, e.g., an electronic machine or a computing system. The risk elements in device 1000 may be related to a drug including elements to identify a drug, identify a source of a drug, and risk elements. Identifying information, about the drug, is stored in device 1000 and may include a product identifier 1001, which can be a national drug code (NDC) 1003, a generic code number 1005, package size 1007, package description 1009, plant 1011, plant suppliers 1013, active pharmaceutical ingredient (API) supplier(s) 1015, contract 1017 and approved for drug manufacturing 1019. The national drug code is a code that is assigned by a governmental agency or a trade organization for a particular drug. The generic drug code 1005 is a code that is assigned by a governmental agency or a trade organization for a particular generic drug. The package size 1007 is the various physical sizes of the generic drug, e.g., active ingredient amount, e.g., milligram, prepackaged amount or the like. The package description 1009 is the type of package that the drug is packaged in. The plant 1011 identifies the plant in which the drug or the API is manufactured. The plant suppliers 1013 identifies the supplier(s) of the components of a drug to the plant 1011 that manufactures the drug. The API suppliers 1015 identifies the supplier(s) of the API component(s) of a drug to the plant 1011 that manufactures the drug.

The risk elements may also include competitive information 1021 for a particular drug. The competitive information 1021 may identify other suppliers or manufacturers of a drug. For example, all of the manufacturers of a drug can be part of the competitive information 1021. This will allow the presently described methods and systems to determine risks associated with other drug manufacturers. The risk elements can further include drug component competitive information 1023 for a particular drug. The drug component competitive information 1023 may identify other suppliers or manufacturers of components for a drug. For example, all of the manufacturers of a drug component can be part of the competitive information 1023. Collecting these data elements allows the methods and systems to determine risks associated with other drug component manufacturers. Each of these data fields relates to a particular drug in contrast to the data of risk element device 123, which relates to the drug vendor. Each of these data fields can be used in the calculation of risk in accordance with methods and systems of the present disclosure.

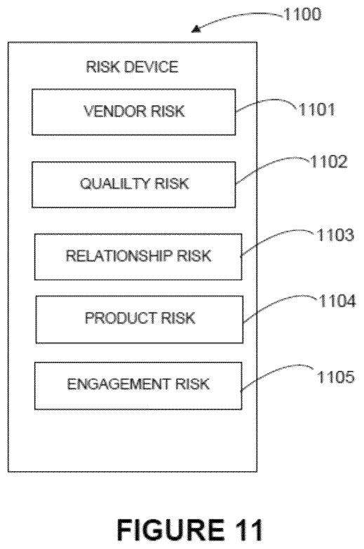

FIG. 11 shows a risk element data storage device 1100 that can receive, store, and calculate risk, e.g., using the risk elements in devices 123, 1000 or in the storage device 114. The device 1100 can be a machine, e.g., an electronic machine or a computing system that includes a memory. Vendor risk 1101 may be risk associated with a particular organization that is involved with the manufacture, packaging, component supply of a drug. The drug may be identified using data from the device 1100 while the vendor may be identified using data from the device 1000. The vendor risk 1101 may be used when establishing pricing in a contract to take into account any problems with supply of a drug. AS discussed herein the established pricing may be the price for a particular drug that is negotiated by the GPO at which the participants may purchase the drug. In an example embodiment, the risk is analyzed before awarding a contract to a vendor/supplier for a particular drug. The vendor risk 1101 may be calculated from a quality risk 1102 and a relationship risk 1103 and reflects the overall risk of a drug supply disruption from the vendor. The quality risk 1102 may be a risk associated with quality of the drug product from the drug vendor. The quality risk is a measure of the likelihood that a drug will meet its promised purity and effective ingredient level. The quality risk 1102 may be calculated from location of plant, certifications of plant, suppliers of ingredients, financial data, governmental regulation compliance data and the like. Relationship risk 1103 may be calculated from compliance with contractual obligations, customer service and transparency and the like. Product risk 1104 is individual for each drug (there may be over 17,000 individual drugs) and represents the risk that a supply of any particular drug will experience a disruption in supply. The product risk 1104 and the relationship risk 1103 may also be calculated as described herein.

Engagement risk 1105 is calculated once the drug production is awarded to a particular vendor and measures the risk of that particular engagement of the vendor. In an example embodiment, the engagement risk 1105 is calculated from a combination of a drug specific risk and a vendor risk, which can be weighted. Weighting can be applying a factor to a value stored in the devices described herein. The factor can be a value between zero and a maximum weight, e.g., one, two, five, ten or the like. The weighting can also be adjusting the value through the use of interactive adjustments, e.g., an input/output device that interacts with the display. By way of example, at a first point in time a drug A with vendor A has an engagement risk A. If the contract for the drug A is changed at a future time for any reason to vendor B, then the engagement risk will change when an organization changes from vendor A to vendor B. In operation, the engagement risk elements can be changed before awarding a drug supply contract between vendor A and vendor B to calculate and show the engagement risk 1105 for both engagements, i.e., vendor A or vendor B.

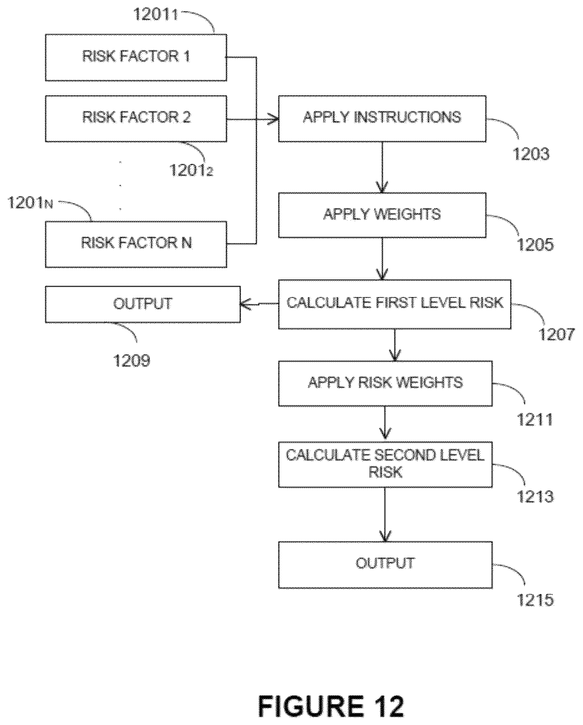

FIG. 12 shows a method 1200 for determining risk in an example embodiment. Multiple risk factors 1-N are acquired at blocks 1201-1-1201-N. The risk factors 1-N can be data (values) provided the vendors, e.g., via surveys, data from the drug supply purchaser, governmental data, regulatory data, user input data, or any other data described herein. Instructions for how the risk factors 1-N are related to any risk are stored, e.g., in a storage device 114. In an example embodiment, the risk factors can be set with associated values in the risk storage device 123 or storage device 114. The risk factors can be in the vendor ratings data 116, the product data 118, data in the regulatory data storage device 125, data in the external sources storage device 127, the contract data 121, the vendor data 129 and/or the internal data 131.

At block 1203, the instructions may be applied to the risk factors. At block 1205, weights are applied to the risk factors. At block 1207, first level risk factors are calculated. First level risk can be the risk that is calculated from the data itself. Subsequent levels may be calculated from the results at prior levels. The first level risk calculation may use the risk factors 1-N from 1201-1-1201-N (data stored in data storage devices), which may be weighted at block 1205. At block 1209, the first level risks are output. Outputting the first level risks may include storing the calculated risks in a storage device, transmitting the risks in machine readable form, displaying the risks on a screen and/or sending an electronic alert to a device associated with a person if a risk exceeds a threshold. The thresholds can be stored as an instruction at block 1203. In an example embodiment, the risk factors can be set with associated values in the risk storage device 123.

At block 1207, the calculated first level risks may be passed for further interpretation. Such a passing can store the data within the storage device 114 and further operations can use this data in further calculations. At block 1209, the first level risks can be output. At block 1211, weights can be applied to the first level risks. At block 1213, second level risks are calculated. At block 1215, the second level risks are outputted. Outputting the second level risks may include storing the calculated risks in the storage device 114, transmitting the risks in machine readable form, displaying the risks on a screen and/or sending an electronic alert to a device associated with a person if a risk exceeds a threshold. The thresholds may then be stored as an instruction at block 1203. The second level risks are thus calculated from previously calculated first level risks. Thus, the second level risk(s) rely on the predicates of the first level risk(s), which are risks that are calculated from the domain of data as described herein relating to the drug supply. Thus, the second level risk(s) are a higher order logic relative to the first level risk(s). The second level risks use at least one variable outcome from the first level risks to determine the second level risk.

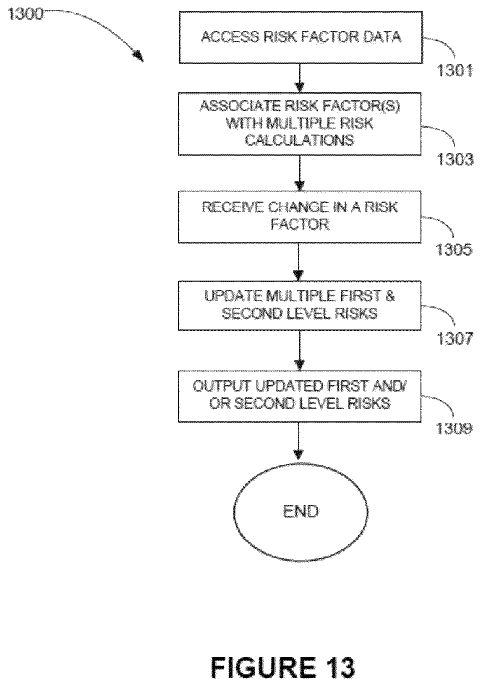

FIG. 13 shows a method 1300 for determining risk in an example embodiment. Multiple risk factors 1301 are accessed. The risk factors 1301 may be data from the vendors, e.g., via surveys, data from the drug supply purchaser, governmental data, regulatory data or any other data described herein. Data associated with the risk factors 1301 may be culled from governmental reports, e.g., by inputting risks associated with reports via manual inputs or via electronically receiving and interpreting data from such reports. The risk factors data at block 1301 may be set and stored in the storage device 114. The risk factors data can be any data or information as described herein.

At block 1303, the risk factors data are associated with one or more than one risk calculations. The risk factors data may be inputs into multiple risk calculations. The risk factors may be weighted in the calculations. The weights assigned to the risk factors may be different between different calculations. The weights allow any one of the risk factor data to have a greater influence on the calculated risk result than other risk factor data.

At block 1305, a change in a risk factor is received. The risk factor change may be from a manual entry into a device that is connected to the storage device 114 or automatically retrieved, such as from one or more databases locally and/or networked. The risk factor may be recalculated using data received from a vendor in response to survey questionnaire or an update to any of the questions posed to a drug vendor. The data can also be entered into the data storage 114 using a device and an application programming interface (API). The risk factor change may be from a public database that feeds data into the present system, e.g., a governmental database, a subscription database, a trade group database, or the like.

The change of risk factors or risk factor data may be from a vendor survey. Such a vendor survey may require the vendor to report its identification code for financial rating companies so that financial data may be retrieved from such financial rating companies. A vendor may be required to report exclusions by a governmental agency from participating in government programs, importation into the country, manufacturing for drugs destined for the country, or otherwise. Manufacturing agreements may also be reported. A factor of manufacturing practices inspection may also be reported, which may include inspection dates, inspection of component suppliers, adherence to good manufacturing process, and payment of Generic Drug User Fee Amendment (GDUFA) fees. Other factors may include overall average, unadjusted order fill rate over a set time period, e.g., four months, six months, or a year; wholesale shipments percentage; method of calculation of order fill rate; sale of Schedule II drugs, capability or desire to package drugs into larger quantity beyond current product catalog; use of healthcare distribution management association (HDMA) format; date ready to test drug; date ready to go live for production; and description of competitive advantage of the vendor. The risk factors further may include data about each manufacturing plant of the vendor, including name, location, country, plant type, last governmental inspection (e.g., FDA), regulatory issues in past year, two years, three years, or five years, was GDUFA fee paid, plant capacity utilized, vendor owned plant, or contractual manufacturing plant. Recall information may be another factor. Supplier diversity information may be another factor. Product portfolio (list of drugs in which the vendor is engaged in manufacturing) may also be included in risk.

At block 1307, the first level risks and the second level risks are recalculated. In an example embodiment, only the first level risks that are based on the changed risk factor data are recalculated. Thereafter, the second level risks, which are those that use the first level risks as inputs, are calculated.

At block 1309, the recalculated first level risk and the recalculated second risk are outputted. Outputting may include visually displaying the results on a display using indicators of the risk. Outputting can also include generating a display, e.g., display 600, 700 or 800 or the like. Outputting may also include storing the risk calculations in a database that is part of a machine memory, e.g., in storage device 114. Outputting may also include sending electronic notifications to devices associated with companies that may be affected by a change in the risk or to individuals that are responsible for making business decisions associated with risk.

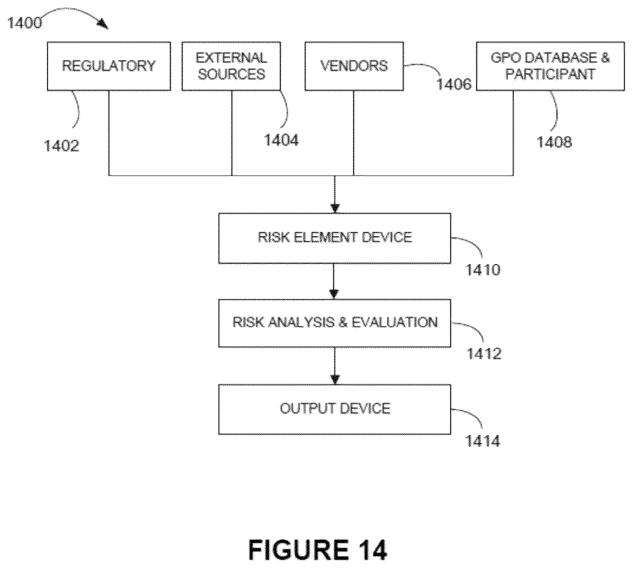

FIG. 14 is a block diagram of an example system 1400, according to an example embodiment. The system 1400 is an example environment in which risks associated with a drug supply may be performed. The system 1400 includes a regulatory device 1402, an external source device 1404, a vendor device 1406, and a purchasing organization device 1408, which may selectively store data or generate data to be used in determining risk. The purchasing organization device 1408 may be used by a group purchasing organization and/or a private label distributor. The regulatory device 1402 may include the regulatory device 108 as described herein. The external device 1404 may include the user device 110 or participant device 112 as described herein. The vendor device 1406 may include the drug vendor device 113 as described herein. The purchasing organization device 1408 may include the analysis device 113 as described herein.

In an example, the devices 1402-1408 may communicate such data to the risk element device 1410. The risk element device 1410 operates to collect the required data for risk analysis. The risk element device 1410 may include a memory to store the data and a processor to organize the data. The risk element device 1410 may request data from the device 1402-1408. The risk element device 1410 provides the data to the risk analysis and evaluation device 1412, which can calculate risk as described herein. When the risk analysis and evaluation device 1412 determines risk, e.g., at any of the first level, the second level, third level and/or the overall risk, the risk analysis and evaluation device 1412 sends the determined risk to an output device 1414. The output device 1414 can organize the data in a form to present on a display, store in a memory, or send a notification.

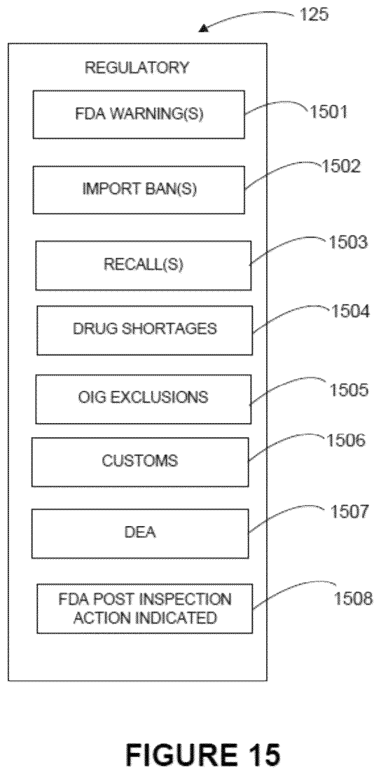

FIG. 15 illustrates the regulatory data storage device 125, which may be stored in the regulatory device 1402 or in the storage device 114, according to example embodiments. The regulatory data storage device 125 may be deployed in the system 100 or 1400 (e.g., the regulatory device 108 or 1402), or may otherwise be used. The regulatory data storage device 125 may be included in computer(s) or memory systems associated with a regulatory agency, e.g., the Federal Drug Administration (FDA), Office of the Inspector General (OIG), the Drug Enforcement Agency (DEA), state regulatory agency, and the like. The regulatory data storage device 125 may include FDA warning data 1501, import ban data 1502, recall data 1503, drug shortage data 1504, OIG exclusion data 1505, customs data 1506, DEA data 1507, and FDA post inspection action indicated data 1508. Any of this data may be used to evaluate risk to a drug supply. FDA post inspection action indicated data 1508 may be used a predictor of future governmental warnings, impart bans or recalls for a particular drug as the data relates to a drug, a vendor or a manufacturer.

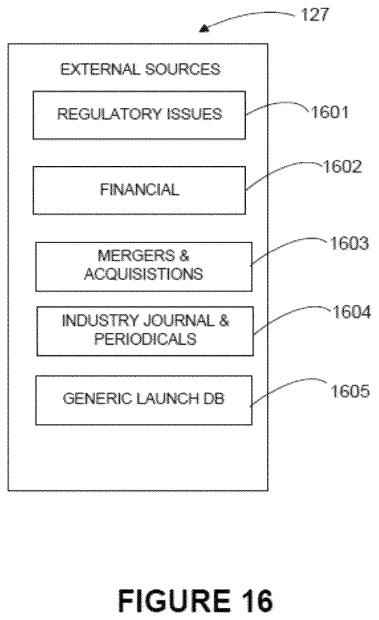

FIG. 16 illustrates the external sources data 127, which may be stored in the external device 1404 or the storage device 114, according to example embodiments. The external sources data 127 may be deployed in the system 100 or 1400 (e.g., the devices 110, 112 or 1404), or may otherwise be used. The external sources data 127 may include computer(s), processors or memory systems associated with external source(s). The storage device 127 can receive data from external sources. An external source device 1404 provides data for determining risk apart from regulatory source or a vendor source. The external source data 127 includes, but is not limited to, regulatory issues 1601, financial issues 1602, mergers and acquisition 1603, industry journal and periodicals 1604, and generic launch database 1605. The regulatory issues 1601 may include regulatory issues related to a drug from a provider or a supplier to a vendor. The regulatory issues 1601 can range from no issues to a warning or possible ban of a drug. The financial issues 1602 can range from a vendor or supplier being on sound financial ground or having financial issues that may indirectly or directly affect the supply of a drug. The mergers and acquisition 1603 relates to announced business mergers or acquisitions related to a vendor or supplier. The industry journals or periodicals 1604 provides data from journals and periodicals, and possibly other industry information sources, that relate to the drug production and distribution. The generic launch database 1605 provides data related to preparing to launch a new source of a drug or increased production of a drug from a vendor. All of this data may be found from multiple, separate and distinct sources. The present systems and methods can pull the data together that is relevant to a drug supply. This data may be used for determining a first level risk or a subsequent level risk.

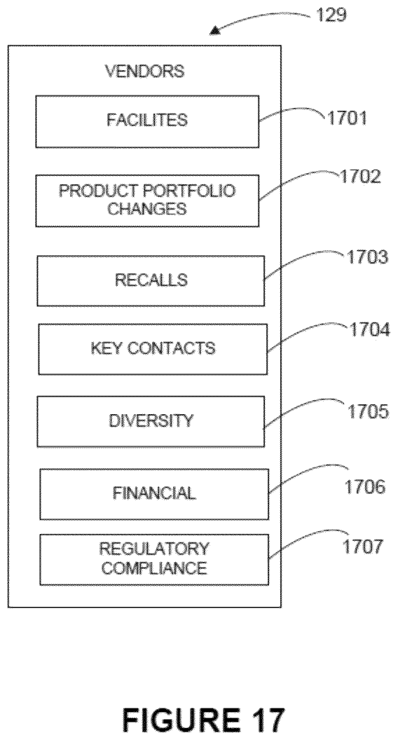

FIG. 17 illustrates the vendors data storage device 129, which may be stored in the vendor device 1406 or the storage device 114, according to example embodiments. The vendors data 129 may be deployed in the system 100 or 1400, or may otherwise be used. The vendors data 129 may be included in computer(s), processors or memory systems associated with an external source(s). The vendors data 129 provides data for a vendor of a drug, which may include facility data 1701, product portfolio change data 1702, recall data 1703, a key contracts data 1704, diversity data 1705, financial data 1706 and regulatory compliance data 1707. The facility data 1701 can be data relating to the facilities that handle the drug in some manner, e.g., storage, manufacture, shipping, etc. The product portfolio change data 1702 may indicate that the vendor is changing the drugs that it handles or manufactures. The recall data 1703 represents the drug recall data, e.g., as required by a governmental agency or initiated by the vendor. The diversity data 1705 may represent the diversity within the vendor or the diversity of sources used by the vendor. The financial data 1706 represents the financials of the vendor. The regulatory compliance data 1707 represents the vendor being in compliance with regulatory compliance requirements of a governmental agency or a purchaser of the drug. This data may be used for determining a first level risk or a subsequent level risk.

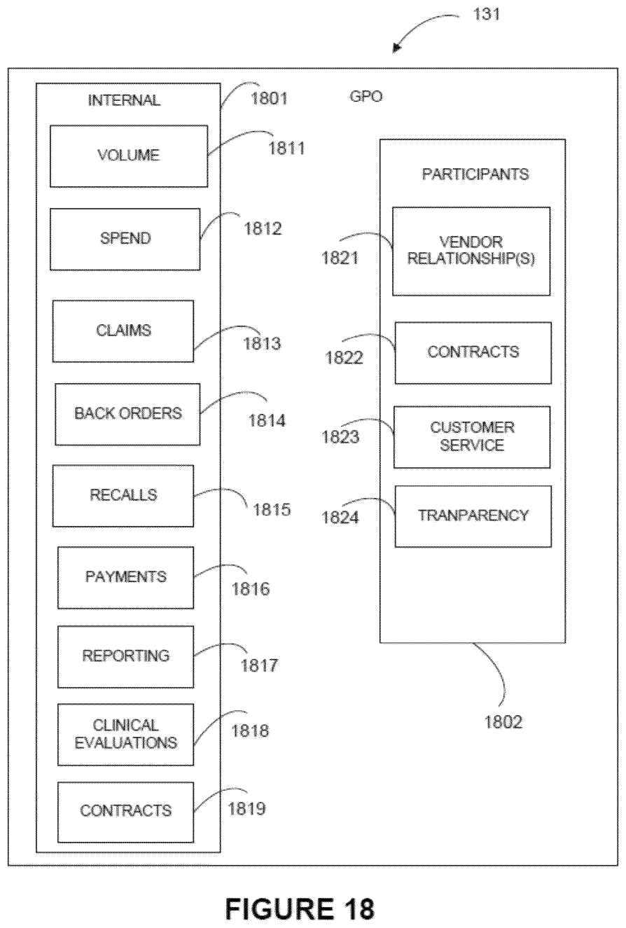

FIG. 18 illustrates the purchasing organization data storage device 131, which may be stored in the purchasing organization device 1408 or the storage device 114, according to an example embodiment. The purchasing organization may be a group purchasing organization and/or a private label distributor. The purchasing organization data 131 may be deployed in the system 100 or 1400 (e.g., the storage device 114), or may otherwise be used. The purchasing organization data 131 may be included in computer(s), processors, or memory systems associated with the organization. A purchasing organization data 131 provides data related to a drug, which may be separated into an internal data collection 1801 and a participant data collection 1802. The internal data collection 1801 includes data relating to internal data in the purchasing organization. The participant data collection 1802 includes data relating to entities providing drugs, particularly, generic drugs. The internal data collection 1801 may include data relating to drug volume 1811, drug spend 1812, drug claims 1813, drug back order 1814, drug recall(s) 1815, payments 1816, reporting 1817, clinical evaluation 1818 and contract(s) 1819. The participant data collection 1802 may include data relating to vendor relationship 1821, contacts 1822, customer service 1823, and transparency 1824. This data can be objective as it relates to quantifiable performance or subjective as it relates to impressions and opinions of personnel of the GPO.

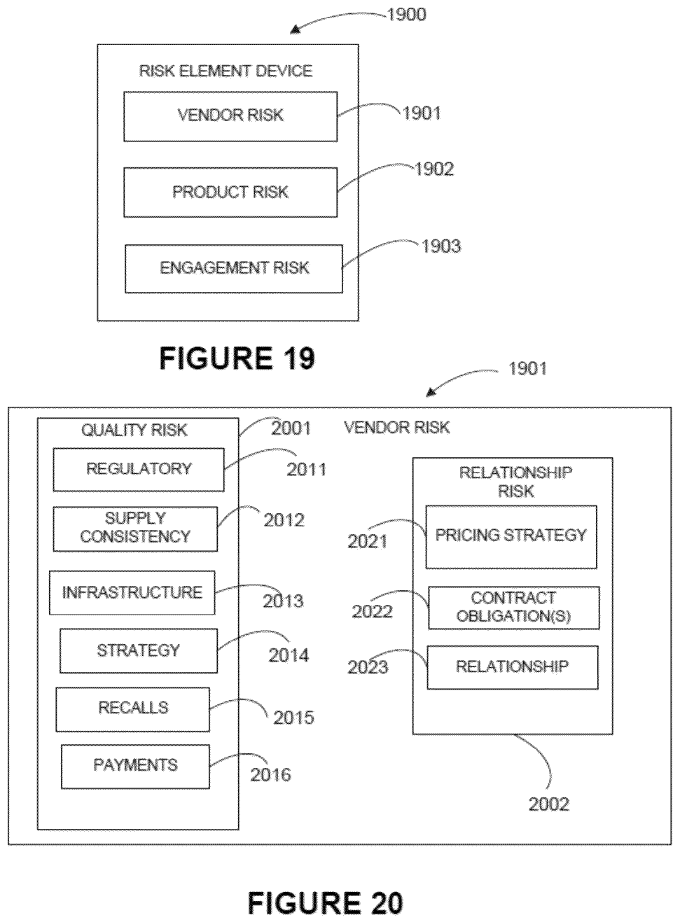

FIG. 19 illustrates a risk element device 1123, according to an example embodiment. The risk element device 1123 may be deployed in the system 100 or 1400 (e.g., the analysis device 102 or user device 110), or may otherwise be used. The risk element device 1123 may include computer(s), processors or memory systems associated with a risk evaluation system. The risk element device 1123 includes a vendor risk subsystem 1901, a product risk subsystem 1902, and an engagement risk subsystem 1903. Each of the subsystems 1901-1903 may include computer(s), processors or memory systems. The vendor risk subsystem 1901 may employ instructions to determine risk associated with a vendor using the data in the system. The product risk subsystem 1902 may employ instructions to determine risk associated with a product, e.g., a drug, generic drug and the like, using the data in the system. The engagement risk subsystem 1903 may employ instructions to determine risk associated with an engagement between a vendor and the group purchasing organization and/or private label distributor using the data in the system.

FIG. 20 illustrates the vendor risk subsystem 1901, according to an example embodiment, in which certain types of data are used to evaluate vendor risk. The vendor risk subsystem 1901 may be deployed in the system 100 or 1400 (e.g., the analysis device 102 or user device 110), or may otherwise be used. The vendor risk subsystem 1901 may include computer(s), processors or memory systems associated with a risk evaluation system. The vendor risk subsystem 1901 includes both quality risk 2001 and relationship risk 2002, which can be stored in storage devices. The quality risk 2001 can be based on a regulatory data 2011, supply consistency data 2012, infrastructure data 2013, strategy data 2014, people data 2015 and financial health data 2016, which all relate to a drug or parts of a drug supply chain. The relationship risk 2002 can be based on a pricing strategy data 2021, contract obligation data 2022, and relationship data 2023. The data may be used to determine either the quality risk 2001 or the relationship 2002 using weighted data in the calculation.

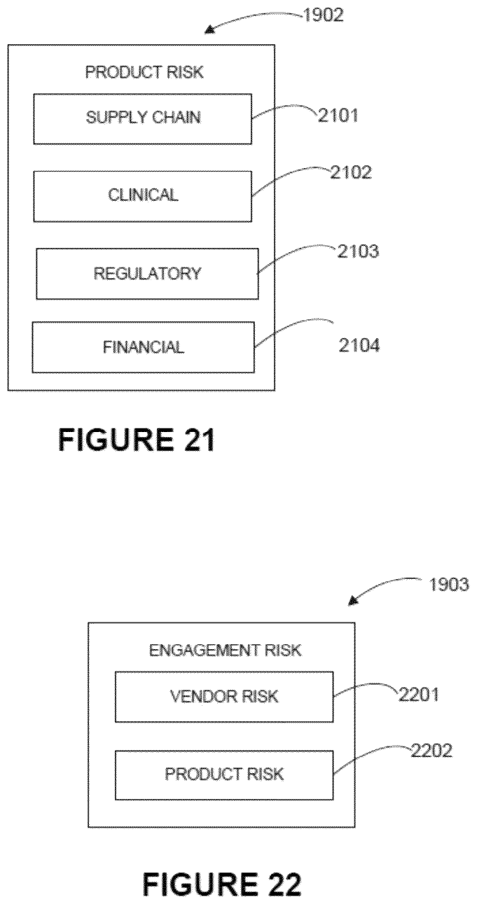

FIG. 21 illustrates the product risk subsystem 1902, according to an example embodiment. The product risk subsystem 1902 may be deployed in the system 100 or 1400 (e.g., the analysis device 102), or may otherwise be used. The product risk subsystem 1902 may include computer(s), processors or memory systems associated with a risk evaluation system. The product risk subsystem 1902 can be based on supply chain data 2101, clinical data 2102, regulatory data 2103, and financial data 2104. This data can be weighted and combined to calculate the product risk.

FIG. 22 illustrates the engagement risk subsystem 1903, according to an example embodiment. The engagement risk subsystem 1903 may be deployed in the system 100 or 1400 (e.g., the analysis device 102 or user device 110), or may otherwise be used. The engagement risk subsystem 1903 may include computer(s), processors or memory systems associated with a risk evaluation system. The engagement risk subsystem 1903 may determine engagement risk using the vendor risk 2201 from the vendor risk device 1901 and the product risk 2202 from the product risk device 1902. This data can be weighted and combined to calculate the engagement risk.

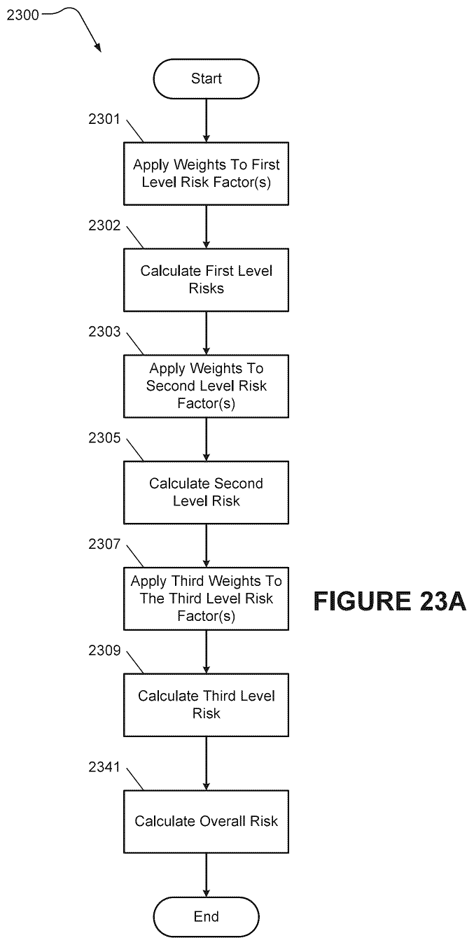

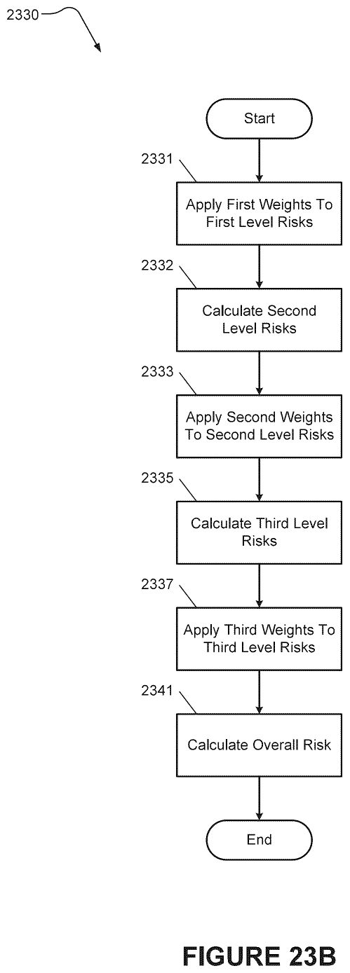

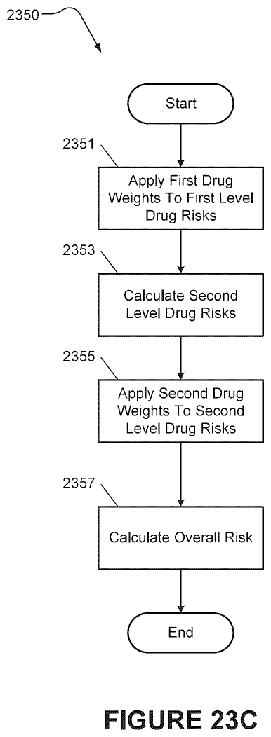

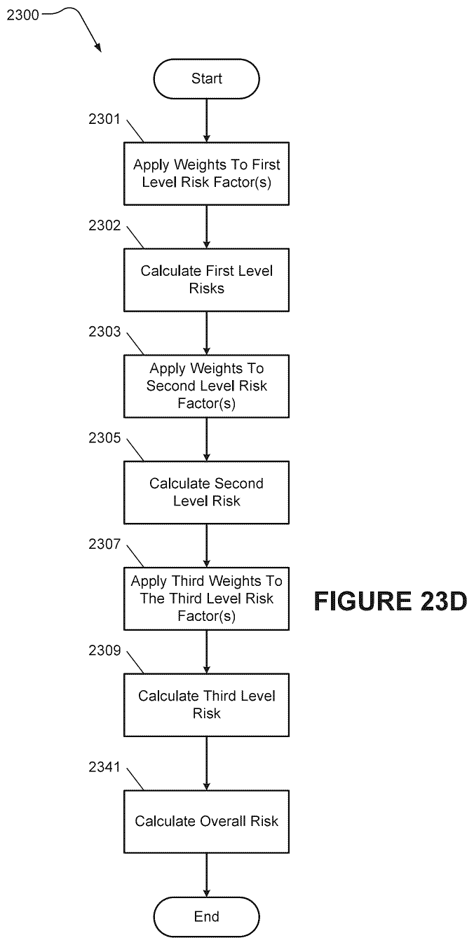

FIG. 23A illustrates a risk analysis and evaluation method 2300, according to an example embodiment. The risk analysis and evaluation method 2300 may be deployed in the system 100 or 1400 to determine the drug supply evaluation or may otherwise be used and may be used to evaluate a risk of a drug supply including both a risk associated with a vendor of a drug and a risk associated with the drug itself. The risk analysis and evaluation method 2300 may include computer(s), processors or memory systems associated with a risk evaluation system. The risk analysis and evaluation method 2300 can be performed, for example, in the analysis device 102 or the user device 110. The risk analysis and evaluation method 2300 may apply weights to first level risk elements 2301. Once the risk elements are weighed, the first level risk is calculated using the weighted risk elements, 2302. At block 2303, weights are applied to second level risk elements, which can include the first level risks and additional data. At block 2305, the second level risk is calculated using the weighted second level risk elements. At block 2307, weights are applied to third level risk elements. At block 2309, the third level risk is calculated using the weighted third level risk elements. At block 2311, overall risk is calculated, e.g., from either the calculated second level risk or the calculated third level risk.

A first level risk can be calculated directly from the data, either weighted or not. The first level risk may include, but are not limited to, governmental issues, e.g., FDA Issues, other regulatory issues (e.g., OIG related issues), product recalls, payment of government fees (e.g., payment of generic drug user fee amendments), plant audit results and the like. The first level risk may include, but are not limited to, vendor issues, e.g., average order fill rate, patient impact, backorder(s), backorder resolution date reliability, state of facilities, manufacturing systems, manufacturing capacity, plant location risk, political risk, disaster recovery, alignment with the purchasing organization, pipeline strength, vertical integration (e.g., API and CRO), core competency, senior management engagement and control of operations, employee training and development plan(s), financial comparative rating, purchasing organization as % of vendor revenue, majority stakeholders, mergers and acquisitions, incentives and contractual enhancements, firm pricing compliance, firm pricing terms, PPD/Admin Fee payment (timelines), reporting issues (timeliness and accuracy), failure to supply adherence, completion and accuracy of supplier surveys, senior management engagement with purchasing organization, national account manager engagement with purchasing organization, customer service, transparency, number of Abbreviated New Drug Application (ANDA) holders, number of active manufacturers of a drug, number of drug master file holders (API), manufacturer's US market share, plant location risk, use of contract manufacturer (e.g., outsourcing), multi-source the active pharmaceutical ingredient (API), vertical integration, inventory at hand, unique place in therapy of drug, disease sensitivity or severity, narrow therapeutic index, product volume, and product spend. The first level risk can include, but are not limited to, regulatory data, e.g., regulatory—API, regulatory—contract manufacturer, and regulatory—other (DEA etc.).

A second level risk is calculated, at least in part, on at least one first level risk. Thus, the second level risk is dependent on the first level risk and is used to further refine the calculated risk into a result on which a decision as to an action related to a vendor may be based. The second level risk may include regulatory risk, which can be based on FDA Issues, other regulatory issues (e.g., issued by the OIG), product recalls, payment of government fees (e.g., payment of generic drug user fee amendments), plant audit results.

The second level risk may include supply consistency risk, which can be based on vendor issues, e.g., average order fill rate, patient impact, backorder(s), backorder resolution date reliability.

The second level risk may include infrastructure risk, which can be based on state of facilities, manufacturing systems, manufacturing capacity, plant location risk, political risk, and disaster recovery.

The second level risk may include strategy risk, which can be based on alignment with the purchasing organization, pipeline strength, vertical integration (e.g., API and CRO), core competency.

The second level risk may include people risk, which can be based on senior management engagement and control of operations, employee training and development plan(s), and the like.

The second level risk may include financial health, which can be based on purchasing organization as % of vendor revenue, majority stakeholders, and mergers and acquisitions.

The second level risk may include pricing strategy, which can be based on incentives and contractual enhancements, firm pricing compliance, and firm pricing terms.

The second level risk may include contract obligations, which can be based on PPD/Admin Fee payment (timelines), reporting issues (timeliness and accuracy), failure to supply adherence, and completion and accuracy of supplier surveys.

The second level risk can include relationship risk, which can be based on senior management engagement with purchasing organization, national account manager engagement with purchasing organization, customer service, and transparency.

The second level risk may include supply chain risk, which can be based on number of Abbreviated New Drug Application (ANDA) holders, number of active manufacturers of a drug, number of drug master file holders (API), manufacturer's US market share, plant location risk, use of contract manufacturer (e.g., outsourcing), multi-source the active pharmaceutical ingredient (API), vertical integration, and inventory at hand.

The second level risk may include clinical risk, which can be based on unique place in therapy of drug, disease sensitivity or severity, and narrow therapeutic index.

The second level risk may include a second regulatory risk, which can be based on the first level risk, which may be combined with regulatory—API, regulatory—contract manufacturer, and regulatory—other (DEA etc.).

The second level risk may include a financial risk, which can be based on product volume and product spend.

Third level risks may include quality risk and relationship risk and be based at least in part on the second level risks. Quality risk can be based on the first regulatory risk, a supply consistency risk, infrastructure risk, strategy risk, people risk, and financial health risk. Relationship risk can be based on the pricing strategy risk, contract obligations, and second relationship risk.

The overall risk may be based on the vendor risk and the product risk. The vendor risk may be based on the quality risk and the relationship risk. The product risk may be based on supply chain risk, clinical risk, regulatory risk, and financial risk.

Each of these calculated risks can be output to a display.

FIG. 23B illustrates a risk analysis and evaluation method 2330, according to an example embodiment. The risk analysis and evaluation method 2330 may be deployed in the system 100 or 1400 to determine a risk associated with a drug vendor (i.e., a vendor of a drug). The risk analysis and evaluation method 2300 may include computer(s), processors or memory systems associated with a risk evaluation system. The risk analysis and evaluation method 2300 can be performed in the analysis device 102 or the user device 110.

The risk analysis and evaluation method 2330 may apply first weight values to first level risks, respectively, at 2331. The first weight values may be predetermined values stored in memory and obtained (e.g., by one or more processors) from the memory. The first level risks may include, for example, a first risk based on regulatory issues of the drug vendor with the U.S. Food and Drug Administration (FDA), a second risk based on regulatory issues of the drug vendor with other regulatory authorities, a third risk based on payment of generic drug user fee amendment (GDUFA) fee payment by the drug vendor. A first weight value, a second weight value, and a third weight value are associated with the first risk, the second risk, and the third risk, respectively. For example only, the first, second, and third weight values may be 80 percent, 5 percent, and 15 percent, respectively, or other suitable values. The sum of the first, second, and third weight values is equal to 100 percent. In various implementations, the first, second, and third risks may be values between 0 and 100 and may be user input, obtained from a database, or obtained in another suitable manner.

The first level risks may also include a fourth risk based on an impact of backorders on patients (e.g., e.g., based on duration and clinical risk), a fifth risk based on a percentage of the drug vendor's product portfolio that is on back order, a sixth risk of a frequency of backorders of the drug vendor, and a seventh risk based on whether an actual delivery leadtime of the drug vendor is less than or equal to a promised leadtime by the drug vendor. A fourth weight value, a fifth weight value, a sixth weight value, and a seventh weight value are associated with the fourth risk, the fifth risk, the sixth risk, and the seventh risk, respectively. For example only, the fourth, fifth, sixth, and seventh weight values may be 30 percent, 30 percent, 20 percent, and 20 percent, respectively, or other suitable values. The sum of the fourth, fifth, sixth, and seventh weight values is equal to 100 percent. In various implementations, the fourth, fifth, sixth, and seventh risks may be values between 0 and 100 and may be user input, obtained from a database, or obtained in another suitable manner.

The first level risks may also include a eighth risk based on how technologically state of art a manufacturing facility of the drug vendor is, a ninth risk based on whether the drug vendor has a good information system that helps to ensure data integrity, a tenth risk based on whether the drug vendor has enough manufacturing capacity, an eleventh risk based on risk in locations where the facilities of the drug vendor are located, a twelfth risk based on the drug vendors disaster recovery redundancies, and a thirteenth risk based on whether the drug vendors employees are trained to follow current good manufacturing practice (CGMP) standards. A eighth weight value, a ninth weight value, a tenth weight value, an eleventh, a twelfth, and a thirteenth weight value are associated with the eighth risk, the ninth risk, the tenth risk, the eleventh risk, the twelfth risk, and the thirteenth risk, respectively. For example only, the eighth, ninth, tenth, eleventh, twelfth, and thirteenth weight values may be 10 percent, 25 percent, 20 percent, percent, 20 percent, and 10 percent, respectively, or other suitable values. The sum of the eighth, ninth, tenth, eleventh, twelfth, and thirteenth weight values is equal to 100 percent. In various implementations, the eighth, ninth, tenth, eleventh, twelfth, and thirteenth risks may be values between 0 and 100 and may be user input, obtained from a database, or obtained in another suitable manner.

The first level risks may also include a fourteenth risk based on how vertically integrated the drug vendor is in terms of an active pharmaceutical ingredient (API), a fifteenth risk based on how vertically integrated the drug vendor is in terms of a clinical research organization (CRO), and a sixteenth risk based on whether the drug vendor has promising product launches planned in the future. A fourteenth weight value, a fifteenth weight value, and a sixteenth weight value is associated with the fourteenth risk, the fifteenth risk, and the sixteenth risk, respectively. For example only, the fourteenth, fifteenth, and sixteenth weight values may be 50 percent, 30 percent, and 20 percent, respectively, or other suitable values. The sum of the fourteenth, fifteenth, and sixteenth weight values is equal to 100 percent. In various implementations, the fourteenth, fifteenth, and sixteenth risks may be values between 0 and 100 and may be user input, obtained from a database, or obtained in another suitable manner.

The first level risks may also include a seventeenth risk based on whether the drug vendor is undertaking mergers and/or acquisitions of other entities. A seventeenth weight value is associated with the seventeenth risk. For example only, the seventeenth weight value may be 100 percent or other suitable values. In various implementations, the seventeenth risk may be a value between and 100 and may be user input, obtained from a database, or obtained in another suitable manner.

The first level risks may also include an eighteenth risk based on the drug vendor's pricing terms and a nineteenth risk based on whether the drug vendor is compliant with its pricing terms. An eighteenth weight value and a nineteenth weight value is associated with the eighteenth risk and the nineteenth risk, respectively. For example only, the eighteenth and nineteenth weight values may be 60 percent and 40 percent, respectively, or other suitable values. The sum of the eighteenth and nineteenth weight values is equal to 100 percent. In various implementations, the eighteenth and nineteenth risks may be values between 0 and 100 and may be user input, obtained from a database, or obtained in another suitable manner.

The first level risks may also include a twentieth risk based on whether the drug vendor is on time with payments, a twenty first risk based on whether the drug vendor failed to supply contract terms, a twenty second risk based on whether the drug vendor submits reports on time, and a twenty third risk based on whether the drug vendor submits supplier surveys on time. A twentieth weight value, a twenty first weight value, a twenty second weight value, and a twenty third weight value is associated with the twentieth risk, the twenty first risk, the twenty second risk, and the twenty third risk, respectively. For example only, the twentieth, twenty first, twenty second, and twenty third weight values may be 25 percent, 25 percent, 25 percent, and 25 percent, respectively, or other suitable values. The sum of the twentieth, twenty first, twenty second, and twenty third weight values is equal to 100 percent. In various implementations, the twentieth, twenty first, twenty second, and twenty third risks may be values between 0 and 100 and may be user input, obtained from a database, or obtained in another suitable manner.

The first level risks may also include a twenty fourth risk based on a relationship between senior management of the drug vendor with a drug group purchasing organization (GPO), such as Econdisc, and/or a private label distributor, a twenty fifth risk based on a relationship between a national account manager of the drug vendor with the drug GPO, a twenty sixth risk based on the drug vendor's customer service, and a twenty seventh risk based on transparency of the drug vendor. A twenty fourth weight value, a twenty fifth weight value, a twenty sixth weight value, and a twenty seventh weight value is associated with the twenty fourth risk, the twenty fifth risk, the twenty sixth risk, and the twenty seventh risk, respectively. For example only, the twenty fourth, twenty fifth, twenty sixth, and twenty seventh weight values may be 25 percent, 25 percent, 25 percent, and 25 percent, respectively, or other suitable values. The sum of the twenty fourth, twenty fifth, twenty sixth, and twenty seventh weight values is equal to 100 percent. In various implementations, the twenty fourth, twenty fifth, twenty sixth, and twenty seventh risks may be values between 0 and 100 and may be user input, obtained from a database, or obtained in another suitable manner.

The first weight values are applied (e.g., by one or more processors) to the first level risks, respectively, at 2331. For example only, the first weight values may be multiplied with the first level risks.

Second level risks are calculated (e.g., by one or more processors) at 2332 based on the first level risks weighted by the first weight values, respectively. The second level risks include, for example, a regulatory risk, a supply consistency risk, an infrastructure risk, a strategy risk, a financial health risk, a pricing strategy risk, a contract obligations risk, and a relationship risk.

The regulatory risk is calculated based on the weighted first, second, and third risks. For example, the regulatory risk may be set based on or equal to a sum of the weighted first, second, and third risks. An example equation for calculating the regulatory risk is as follows:

RR=r1*w1+r2*w2+r3*w3,

where RR is the regulatory risk, r1 is the first risk, w1 is the first weight value, r2 is the second risk, w2 is the second weight value, r3 is the third risk, and w3 is the third weight value. In the equation above and the equations that follow, a risk multiplied by a weight value may be considered a weighted version of that risk (i.e., a weighted risk).

The supply consistency risk is calculated based on the weighted fourth, fifth, sixth, and seventh risks. For example, the supply consistency may be set based on or equal to a sum of the weighted fourth, fifth, sixth, and seventh risks. An example equation for calculating the supply consistency risk is as follows:

SC=r4*w4+r5*w5+r6*w6+r7*w7,

where SC is the supply consistency risk, r4 is the fourth risk, w4 is the fourth weight value, r5 is the fifth risk, w5 is the fifth weight value, r6 is the sixth risk, w6 is the sixth weight value, r7 is the seventh risk, and w7 is the seventh weight value.

The infrastructure risk is calculated based on the weighted eighth, ninth, tenth, eleventh, twelfth, and thirteenth risks. For example, the infrastructure may be set based on or equal to a sum of the weighted eighth, ninth, tenth, eleventh, twelfth, and thirteenth risks. An example equation for calculating the infrastructure risk is as follows:

I=r8*w8+r9*w9+r10*w10+r11*w11+r12*w12+r13*w13,

where I is the infrastructure risk, r8 is the eighth risk, w8 is the eighth weight value, r9 is the ninth risk, w9 is the ninth weight value, r10 is the tenth risk, w10 is the tenth weight value, r11 is the eleventh risk, w11 is the eleventh weight value, r12 is the twelfth risk, w12 is the twelfth weight value, r13 is the thirteenth risk, and w13 is the thirteenth weight value.

The strategy risk is calculated based on the weighted fourteenth, fifteenth, and sixteenth risks. For example, the strategy risk may be set based on or equal to a sum of the weighted fourteenth, fifteenth, and sixteenth risks. An example equation for calculating the strategy risk is as follows:

S=r14*w14+r15*w15+r16*w16,

where S is the strategy risk, r14 is the fourteenth risk, w14 is the fourteenth weight value, r15 is the fifteenth risk, w15 is the fifteenth weight value, r16 is the sixteenth risk, and w16 is the sixteenth weight value.

The financial health risk is calculated based on the weighted seventeenth risk. For example, the financial health risk may be set based on or equal to the weighted seventeenth risk. An example equation for calculating the financial health risk is as follows:

FH=r17*w17,

where FH is the financial health risk, r17 is the seventeenth risk, and w17 is the seventeenth weight value.

The pricing strategy risk is calculated based on the weighted eighteenth, and nineteenth risks. For example, the pricing strategy risk may be set based on or equal to a sum of the weighted eighteenth and nineteenth risks. An example equation for calculating the pricing strategy risk is as follows:

PS=r18*w18+r19*w19,

where PS is the pricing strategy risk, r18 is the eighteenth risk, w18 is the eighteenth weight value, r19 is the nineteenth risk, and w19 is the nineteenth weight value.

The contract obligations risk is calculated based on the weighted twentieth, twenty first, twenty second, and twenty third risks. For example, the contract obligations risk may be set based on or equal to a sum of the weighted twentieth, twenty first, twenty second, and twenty third risks. An example equation for calculating the contract obligations risk is as follows:

CO=r20*w20+r21*w21+r22*w22+r23*w23,

where CO is the contract obligation risk, r20 is the twentieth risk, w20 is the twentieth weight value, r21 is the twenty first risk, w21 is the twenty first weight value, r22 is the twenty second risk, w22 is the twenty second weight value, r23 is the twenty third risk, and w23 is the twenty third weight value.

The relationship risk is calculated based on the weighted twenty fourth, twenty fifth, twenty sixth, and twenty seventh risks. For example, the relationship risk may be set based on or equal to a sum of the weighted twenty fourth, twenty fifth, twenty sixth, and twenty seventh risks. An example equation for calculating the relationship risk is as follows:

R=r24*w24+r25*w25+r26*w26+r27*w27,

where R is the relationship risk, r24 is the twenty fourth risk, w24 is the twenty fourth weight value, r25 is the twenty fifth risk, w25 is the twenty fifth weight value, r26 is the twenty sixth risk, w26 is the twenty sixth weight value, r27 is the twenty seventh risk, and w27 is the twenty seventh weight value.

Second weight values are applied (e.g., by one or more processors) to the second level risks, respectively, at 2333. For example only, the second weight values may be multiplied with the second level risks. The second weight values may be predetermined values stored in memory and obtained (e.g., by one or more processors) from the memory

The second weight values include a twenty eighth weight value associated with the regulatory risk, a twenty ninth weight value associated with the supply consistency risk, a thirtieth weight value associated with the infrastructure risk, a thirty first weight value associated with the strategy risk, a thirty second weight value associated with the financial health risk, a thirty third weight value associated with the pricing strategy risk, a thirty fourth weight value associated with the contract obligations risk, and thirty fifth weight value associated with the relationship risk.

For example only, the twenty eighth, twenty ninth, thirtieth, thirty first, and thirty second weight values may be 45 percent, 30 percent, 18 percent, 5 percent, and 2 percent, respectively, or other suitable values. The sum of the twenty eighth, twenty ninth, thirtieth, thirty first, and thirty second weight values is equal to 100 percent. For example only, the thirty third, thirty fourth, and thirty fifth weight values may be 30 percent, 40 percent, and 30 percent, respectively, or other suitable values. The sum of the thirty third, thirty fourth, and thirty fifth weight values is equal to 100 percent.

Third level risks are calculated (e.g., by one or more processors) at 2335 based on the second level risks weighted by the second weights, respectively. The third level risks include a vendor quality risk and a vendor relationship risk.

The vendor quality risk is calculated based on the weighted regulatory risk, the weighted supply consistency risk, the weighted infrastructure risk, the weighted strategy risk, and the weighted financial health risk. For example, the vendor quality risk may be set based on or equal to a sum of the weighted regulatory risk, the weighted supply consistency risk, the weighted infrastructure risk, the weighted strategy risk, and the weighted financial health risk. An example equation for calculating the vendor quality risk is as follows:

VQR=RR*w28+SC*w29+/*w30+S*w31+FH*w32,

where VQR is the vendor quality risk, RR is the regulatory risk, w28 is the twenty eighth weight value, SC is the supply consistency risk, w29 is the twenty ninth weight value, I is the infrastructure risk, w30 is the thirtieth weight value, S is the strategy risk, w31 is the thirty first weight value, FH is the financial health risk, and w32 is the thirty second weight value.

The vendor relationship risk is calculated based on the weighted pricing strategy risk, the weighted contract obligations risk, and the weighted relationship risk. For example, the vendor relationship risk may be set based on or equal to a sum of the weighted pricing strategy risk, the weighted contract obligations risk, and the weighted relationship risk. An example equation for calculating the vendor relationship risk is as follows:

VRR=PS*w33+CO*w34+R*w35,

where VRR is the vendor relationship risk, PS is the pricing strategy risk, w33 is the thirty third weight value, CO is the contract obligations risk, w34 is the thirty fourth weight value, R is the relationship risk, w35 is the thirty fifth weight value.

Third weight values are applied (e.g., by one or more processors) to the third level risks, respectively, at 2337. For example only, the third weight values may be multiplied with the third level risks. The third weight values may be predetermined values stored in memory and obtained (e.g., by one or more processors) from the memory

The third weight values include a thirty sixth weight value associated with the vendor quality risk and a thirty seventh weight value associated with the vendor relationship risk. For example only, the thirty sixth weight value and the thirty seventh weight value may be may be 80 percent and 20 percent, respectively, or other suitable values. The sum of the thirty sixth and thirty seventh weight values is equal to 100 percent.

An overall risk associated with the drug vendor is calculated (e.g., by one or more processors) at 2341 based on the third level risks weighted by the third weight values, respectively. For example, the overall risk may be set based on or equal to a sum of the weighted vendor quality risk and the weighted vendor relationship risk. An example equation for calculating the overall risk of a drug vendor is as follows:

OR=VQR*w36+VRR*w37,

where OR is the overall risk of the drug vendor, VQR is the vendor quality risk, w36 is the thirty sixth weight value, VRR is the vendor relationship risk, and w37 is the thirty seventh weight value.

One, more than one, or all of the first level risks, the second level risks, the third level risks, and the overall risk can be displayed (e.g., by one or more processors) on a display.

FIG. 23C illustrates a risk analysis and evaluation method 2350, according to an example embodiment. The risk analysis and evaluation method 2350 may be deployed in the system 100 or 1400 to determine a risk associated with a drug (a product). The risk analysis and evaluation method 2350 may include computer(s), processors or memory systems associated with a risk evaluation system. The risk analysis and evaluation method 2350 can be performed in the analysis device 102 or the user device 110.

The risk analysis and evaluation method 2350 may apply first drug weight values to first level drug risks, respectively, at 2351. The first drug weight values may be predetermined values stored in memory and obtained (e.g., by one or more processors) from the memory.

The first level drug risks may include, for example, a first drug risk based on a regulatory status of the finished dose facility where the drug is manufactured, a second drug risk based on a regulatory status of the facility where the active pharmaceutical ingredient (API) of the drug is produced, and a third drug risk based on regulatory issues related to the drug.

The first drug weight values include a first drug weight value, a second drug weight value, and a third drug weight value associated with the first drug risk, the second drug risk, and the third drug risk, respectively. For example only, the first, second, and third drug weight values may be 45 percent, 45 percent, and 10 percent, respectively, or other suitable values. The sum of the first, second, and third drug weight values is equal to 100 percent. In various implementations, the first, second, and third drug risks may be values between 0 and 100 and may be user input, obtained from a database, or obtained in another suitable manner.

The first level drug risks may also include a fourth drug risk based on a number of different suppliers there are for the API of the drug, a fifth drug risk based on a number of different active finished dose manufacturers there are of the drug, a sixth drug risk based on whether a small number (e.g., two or less) suppliers of the drug have a market share of greater than a predetermined percentage (e.g., 90 percent) for this drug, and a seventh drug risk based on whether a vendor of this drug has multiple sources of the API of the drug. The first drug weight values include a fourth drug weight value, a fifth drug weight value, a sixth drug weight value, and a seventh drug weight value associated with the fourth drug risk, the fifth drug risk, the sixth drug risk, and the seventh drug risk, respectively. For example only, the fourth, fifth, sixth, and seventh drug weight values may be 30 percent, 30 percent, 15 percent, and 25 percent, respectively, or other suitable values. The sum of the fourth, fifth, sixth, and seventh drug weight values is equal to 100 percent. In various implementations, the fourth, fifth, sixth, and seventh drug risks may be values between 0 and 100 and may be user input, obtained from a database, or obtained in another suitable manner.

The first level drug risks may also include an eighth drug risk based on a narrow therapeutic index (NTI) of the drug, a ninth drug risk based on a disease sensitivity or severity (DSS) associated with the drug, and a tenth drug risk based on a unique place in therapy of the drug. The first drug weight values include an eighth drug weight value, a ninth drug weight value, and a tenth drug weight value associated with the eighth drug risk, the ninth drug risk, and the tenth drug risk, respectively. For example only, the eighth, ninth, and tenth drug weight values may be 50 percent, 25 percent, and 25 percent, respectively, or other suitable values. The sum of the eighth, ninth, and tenth drug weight values is equal to 100 percent. In various implementations, the eighth, ninth, and tenth drug risks may be values between 0 and 100 and may be user input, obtained from a database, or obtained in another suitable manner.

The first level drug risks may also include an eleventh drug risk based on a total volume of the drug purchased by the GPO (e.g., Econdisc) and a twelfth drug risk based on a total amount spend on the drug by the GPO. The first drug weight values include an eleventh drug weight value and a twelfth drug weight value associated with the eleventh drug risk and the twelfth drug risk, respectively. For example only, the eleventh and twelfth drug weight values may be 50 percent and 50 percent, respectively, or other suitable values. The sum of the eleventh and twelfth drug weight values is equal to 100 percent. In various implementations, the eleventh and twelfth drug risks may be values between 0 and 100 and may be user input, obtained from a database, or obtained in another suitable manner.

The first drug weight values are applied (e.g., by one or more processors) to the first level drug risks, respectively, at 2351. For example only, the first drug weight values may be multiplied with the first level drug risks.

Second level drug risks are calculated (e.g., by one or more processors) at 2353 based on the first level drug risks weighted by the first drug weight values, respectively. The second level drug risks include, for example, a drug regulatory risk, a drug supply chain risk, a drug clinical risk, and a drug financial risk.

The drug regulatory risk is calculated based on the weighted first, second, and third drug risks. For example, the drug regulatory risk may be set based on or equal to a sum of the weighted first, second, and third drug risks. An example equation for calculating the regulatory risk is as follows:

DRR=dr1*dw1+dr2*dw2+dr3*dw3,

where DRR is the drug regulatory risk, dr1 is the first drug risk, dw1 is the first drug weight value, dr2 is the second drug risk, dw2 is the second drug weight value, dr3 is the third drug risk, and dw3 is the third drug weight value.

The drug supply chain risk is calculated based on the weighted fourth, fifth, sixth, and seventh drug risks. For example, the drug supply chain risk may be set based on or equal to a sum of the weighted fourth, fifth, sixth, and seventh drug risks. An example equation for calculating the drug supply chain risk is as follows:

DSC=dr4*dw4+dr5*dw5+dr6*dw6+dr7*dw7,

where DSC is the drug supply chain risk, dr4 is the fourth drug risk, dw4 is the fourth drug weight value, dr5 is the fifth drug risk, dw5 is the fifth drug weight value, dr6 is the sixth drug risk, dw6 is the sixth drug weight value, dr7 is the seventh drug risk, and dw7 is the seventh drug weight value.

The drug clinical risk is calculated based on the weighted eighth, ninth, and tenth drug risks. For example, the drug clinical may be set based on or equal to a sum of the weighted eighth, ninth, and tenth drug risks. An example equation for calculating the drug clinical risk is as follows:

DCR=dr8*dw8+dr9*dw9+dr10*dw10,

where DCR is the drug clinical risk, dr8 is the eighth drug risk, dw8 is the eighth drug weight value, dr9 is the ninth drug risk, dw9 is the ninth drug weight value, dr10 is the tenth drug risk, and dw10 is the tenth drug weight value.

The drug financial risk is calculated based on the weighted eleventh and twelfth drug risks. For example, the drug financial risk may be set based on or equal to a sum of the weighted eleventh and twelfth drug risks. An example equation for calculating the drug financial risk is as follows:

DFR=dr11*dw11+dr12*dw12,

where DFR is the drug financial risk, dr11 is the eleventh drug risk, dw11 is the eleventh drug weight value, dr12 is the twelfth drug risk, and dw12 is the twelfth drug weight value.

Second drug weight values are applied (e.g., by one or more processors) to the second level drug risks, respectively, at 2355. For example only, the second drug weight values may be multiplied with the second level drug risks. The second drug weight values may be predetermined values stored in memory and obtained (e.g., by one or more processors) from the memory

The second drug weight values include a thirteenth drug weight value associated with the drug regulatory risk, a fourteenth drug weight value associated with the drug supply chain risk, a fifteenth drug weight value associated with the drug clinical risk, and a sixteenth drug weight value associated with the drug financial risk. For example only, the thirteenth, fourteenth, fifteenth, and sixteenth drug weight values may be 40 percent, 25 percent, 25 percent, and 10 percent, respectively, or other suitable values. The sum of the fourteenth, fifteenth, sixteenth, and seventeenth drug weight values is equal to 100 percent.

An overall risk associated with the drug is calculated (e.g., by one or more processors) at 2357 based on the second level drug risks weighted by the second drug weight values, respectively. For example, the overall risk may be set based on or equal to a sum of the weighted drug regulatory risk, the weighted drug supply chain risk, the weighted drug clinical risk, and the weighted drug financial risk. An example equation for calculating the overall risk of the drug is as follows:

ORD=DRR*dw13+DSC*dw14+DCR*dw15+DFR*dw16,

where ORD is the overall risk of the drug, DRR is the drug regulatory risk, dw13 is the thirteenth drug weight value, DSC is the drug supply chain risk, dw14 is the fourteenth drug weight value, DCR is the drug clinical risk, dw15 is the fifteenth drug weight value, DFR is the drug financial risk, and dw16 is the fifteenth drug weight value.

One, more than one, or all of the first level drug risks, the second level drug risks, and the overall risk of the drug can be displayed (e.g., by one or more processors) on a display.

FIG. 23D illustrates a risk analysis and evaluation method 2370, according to an example embodiment. The risk analysis and evaluation method 2370 may be deployed in the system 100 or 1400 to determine a risk associated with a drug (a product). The risk analysis and evaluation method 2370 may include computer(s), processors or memory systems associated with a risk evaluation system. The risk analysis and evaluation method 2370 can be performed in the analysis device 102 or the user device 110.

At 2371, the overall risk associated with a drug (ORD) and the overall risk of a drug vendor (OR) of that drug are calculated, as described above. At 2373, the risk analysis and evaluation method 2370 includes applying overall weight values to the overall risk associated with the drug and the overall risk associated with the drug vendor of that drug. The overall weight values may be predetermined values stored in memory and obtained (e.g., by one or more processors) from the memory.

The overall weight values include a first overall weight value and a second overall weight value associated with the overall risk associated with the drug and the overall risk associated with the drug vendor of that drug, respectively. For example only, the first and second overall weight values may be percent and 50 percent, respectively, or other suitable values. The sum of the first and second overall weight values is equal to 100 percent. In various implementations, the overall risk associated with the drug and the overall risk associated with the drug vendor of the drug may be values between 0 and 100, as discussed above.

An overall (engagement) risk associated with entering into an engagement (e.g., contract) with the drug vendor for the drug is calculated (e.g., by one or more processors) at 2375 based on the overall risks weighted by the overall weight values, respectively. For example, the overall (engagement) risk may be set based on or equal to a sum of the weighted overall risk associated with the drug and the weighted overall risk associated with the drug vendor of the drug. An example equation for calculating the overall (engagement) risk is as follows:

OER=ODR*ow1+OR*ow2,

where OCR is the overall (engagement) risk associated with entering into an engagement with the drug vendor for supplying the drug, ORD is the overall risk of the drug, ow1 is the first overall weight value, OR is the overall risk associated with the drug vendor, and ow2 is the second overall weight value.

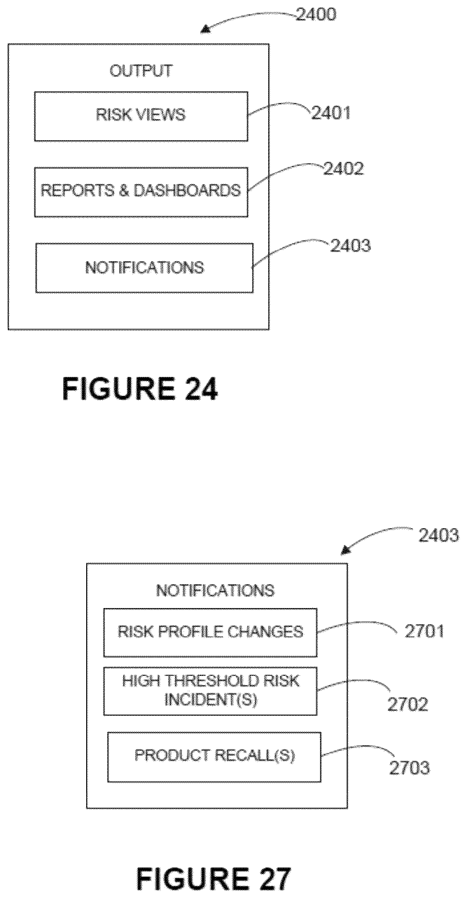

FIG. 24 illustrates the output subsystem 2400, according to an example embodiment. The output subsystem 2400 may be deployed in the system 100 or 1400 (e.g., in the participant device, the user device or the analysis device), or may otherwise be used. The output subsystem 2400 may include computer(s), processors, memory systems, electronic communication subsystem and a display associated with a risk evaluation system. The output subsystem 2400 may include a risk views device 2401, a reports and dashboards device 2402 and a notifications device 2403. Risk views from device 2401 can be shown on a display or included in an electronic message. The risk views from device 2401 can be calculated from the risk analysis and evaluation module 2300, engagement risk from engagement risk module 1903, product risk from the product risk module 1902, vendor risk from the vendor risk module 1901, e.g., on a display as either multiple risks or a notification that a risk has changed to a higher risk. The output subsystem 2400 can show the risks as values or tiers. The reports and notifications module 2402 may show complied reports related to any risk or data as described herein and dashboards related to any risk or data as described herein. The output subsystem 2400 may further send electronic messages to portable electronic device, which includes a display to show the risk.

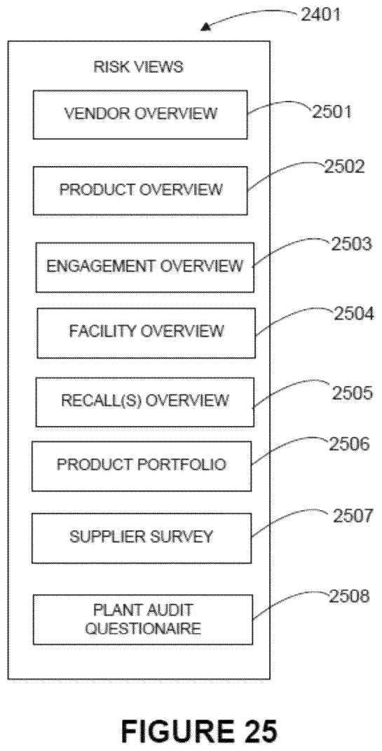

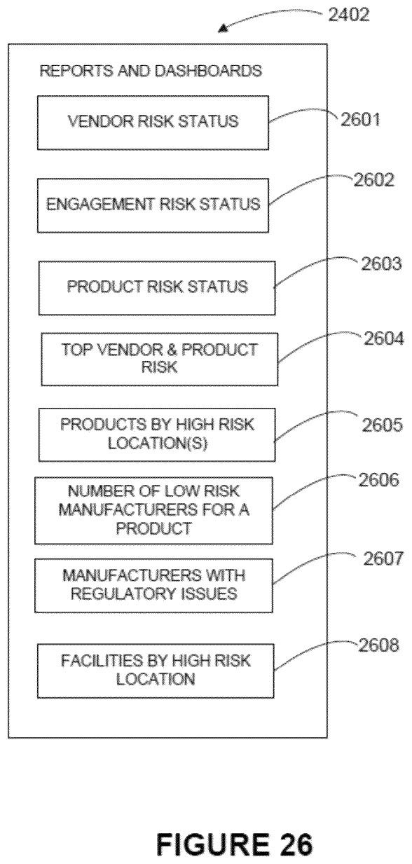

FIG. 25 illustrates the risk views device 2401, according to an example embodiment. The risk views module 2401 may include computer(s), processors, memory systems, electronic communication subsystem and a display associated with a risk evaluation system. The risk views module 2401 may assemble multiple overviews that graphically display the calculated risk values in a form that allows a user to quickly and accurately assess risk. The risk views module 2401 may display, among other things, vendor overview 2501, product overview 2502, engagement overview 2503, facility overview 2504, recalls overview 2505, product portfolio 2506, supplier survey 2507, and plant audit questionnaire 2508. These overviews can show data and calculated risk based on the different organizational classes. For example, the calculated risks are not shown to the vendor. However, a plant audit questionnaire 2508 may be shown to the vendor that relies on that plant or operates that plant. Thus, a classification flag is associated with each of the outputs for display. A device or an operator of the device must meet the classification in order to receive or to see a display of the data or calculations. FIG. 26 illustrates the reports and dashboards subsystem 2402, according to an example embodiment. The reports and dashboards subsystem 2402 may be deployed in the system 100 or 1400 (e.g., in the participant device, the user device or the analysis device), or may otherwise be used. The reports and dashboards subsystem 2402 may include computer(s), processors, memory systems, electronic communication subsystem and a display associated with a risk evaluation system. The reports and dashboards subsystem 2402 may display vendor risk status 2601, engagement risk status 2602, product risk status 2603, top vendor and product risk 2604, products by high risk location(s) 2605, number of low risk manufacturers for a product 2606, manufacturer(s) with regulatory issues 2607, and facilities by high risk location 2608. These reports and dashboards show different organizations of data and calculated risks by different organizational classes. For example, the vendor risk status is not shown to the vendor. In an example, this data is restricted from view by all vendors. The classification of this data may be restricted to devices or operators of devices associated with the GPO. Thus, a classification flag is associated with each of the outputs for display that restricts displaying data only to the GPO, in an example embodiment. A device or an operator of the device must meet the classification in order to receive or to see a display of the data or calculations.

FIG. 27 illustrates the notification subsystem 2403, according to an example embodiment. The notification subsystem 2403 may be deployed in the system 100 or 1400 (e.g., in the participant device, the user device or the analysis device), or may otherwise be used. The notification subsystem 2403 may include computer(s), processors, memory systems, electronic communication subsystem and a display associated with a risk evaluation system or drug supply evaluation system 100. The notification subsystem 2403 may provide electronic notifications to designated devices of personnel in the purchasing organization, e.g., a user device 110, a participant device 112, or a mobile device in communication with any of the devices 110, 112. The electronic notifications produced by the notifications subsystem 2403 can be notifications related to risk determined as described herein. The notifications allow the system 100, 1400 to timely, e.g., in essentially real-time, alerting personnel that risk has changed. The electronic notifications 2403 may include a risk profile change module 2701 that can generate a notification based on a change in the risk associated with a risk profile that includes a calculated risk as described herein. A risk profile may reflect all of the risks associated with a particular drug or a vendor. The electronic notifications 2403 may include a high threshold risk incident module 2702 that can generate a notification based on a change in data elements input into system. When the input data element exceeds a stored threshold value, then a notification is generated and sent. The risks associated with data element that exceeded the threshold changed can then be recalculated or reviewed, e.g., essentially in real-time. The electronic notifications 2403 may include a product recall module 2703 that can generate a notification based on a product recall for a particular drug. The risks associated with product recall may then be recalculated or reviewed, e.g., essentially in real-time. This will allow the GPO or participants in the GPO to react to a change in risk as a potential drug supply risk changes. The GPO can determine is other sources are available, move to a less preferred source, change contract terms, or other related actions.

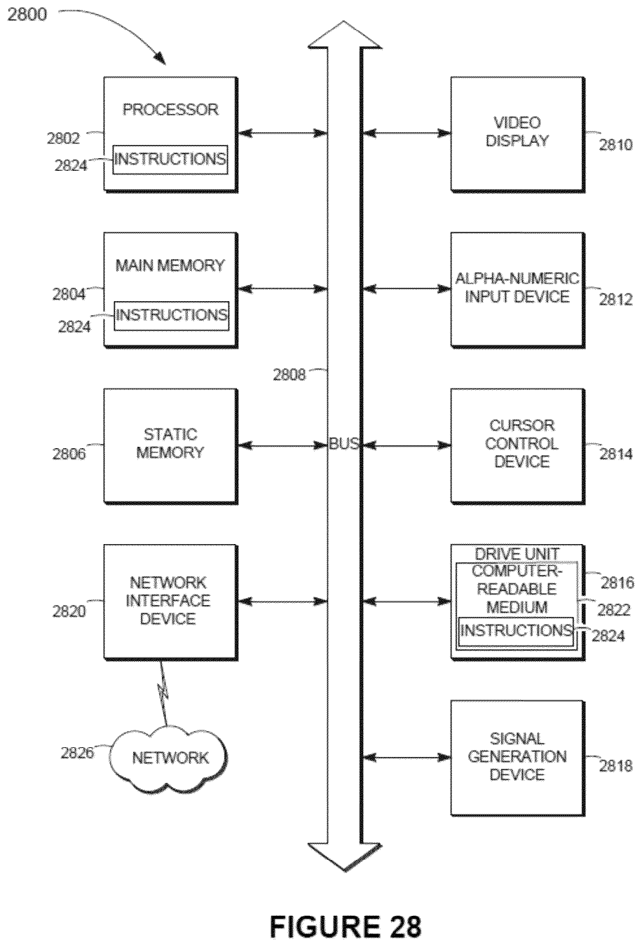

FIG. 28 shows a block diagram of a machine in the example form of a computer system 2800 within which a set of instructions may be executed causing the machine to perform any one or more of the methods, processes, operations, or methodologies discussed herein. The analysis device 102, the governmental regulatory device 108, the user device 110, the participant device 112, may include the functionality of the one or more computer systems 2800.

In an example embodiment, the machine operates as a standalone device or may be connected (e.g., networked) to other machines. In a networked deployment, the machine may operate in the capacity of a server or a client machine in server-client network environment, or as a peer machine in a peer-to-peer (or distributed) network environment.

The example computer system 2800 includes a processor 2802 (e.g., a central processing unit (CPU), a graphics processing unit (GPU) or both), a main memory 2804 and a static memory 2806, which communicate with each other via a bus 2808. The computer system 2800 further includes a video display unit 2810 (e.g., a flat panel, e-ink display, a liquid crystal display (LCD) or a cathode ray tube (CRT)). The computer system 2800 also includes an alphanumeric input device 2812 (e.g., a keyboard), a cursor control device 2814 (e.g., a mouse), a drive unit 2816, a signal generation device 2818 (e.g., a speaker) and a network interface device 2820. The display unit 2810 is capable of displaying the display of risk and data as described herein. In an example, a calculated risk is displayed on the display unit 2810. The

The drive unit 2816 includes a computer-readable medium 2822 on which is stored one or more sets of instructions (e.g., software 2824) embodying any one or more of the methodologies or functions described herein. The instructions (a program or software 2824) may also reside, completely or at least partially, within the main memory 2804 and/or within the processor 2802 during execution thereof by the computer system 2800, the main memory 2804 and the processor 2802 also constituting non-transitory computer-readable media.

The instructions (software 2824) may further be transmitted or received over a network 2826 via the network interface device 2820.

While the computer-readable medium 2822 is shown in an example embodiment to be a single medium, the term “computer-readable medium” should be taken to include a single medium or multiple media (e.g., a centralized or distributed database (e.g., in storage device 114, and/or associated caches and servers) that store the one or more sets of instructions. The term “computer-readable medium” shall also be taken to include any medium that is capable of storing or encoding a set of instructions for execution by the machine and that cause the machine to perform any one or more of the methodologies of the present invention. The term “computer-readable medium” shall accordingly be taken to include, but not be limited to, solid-state memories, and optical media, and magnetic media. In some embodiments, the computer-readable medium is a non-transitory computer-readable medium. In some embodiments, the term “computer-readable medium” can be any medium that has a signal that can be read or interpreted by a computing machine.

The device or computing system 2800 can calculate the indicators of the vendor performance, including risk. The system 2800 can further allow the user to change some values and then recalculate the vendor performance. The computing system 2800 can generate and update any of the displays shown herein, e.g., FIGS. 6-8.

The computing system 2800 may use at least one weight in calculating the risk for a vendor. The computing system 2800 may further produce a display to visually highlight with an indicator of the risk.

The indicators on a display can provide visual indications of risk. Examples of indicators may be color or size. Other indicators that highlight the risk in a machine to human interface are within the scope of the present disclosure. In an example, the computing system 2800 may change the size of the indicators for a higher risk or may change the font. In an example, the computing system 2800 may change the location of the data for a higher risk or a risk above a threshold on the display. The indicators can be varied based on the degree of failure to meet the risk threshold.

In an embodiment, the indicators can include varied colored fonts to indicate that measures have met the threshold, are at the threshold or have failed the threshold. That is colors may be used to indicate the risk. Also, the position of the indicator, e.g., on a graph (line, bar, dial, etc.) may also provide a visual indicator of the risk.

Other types of rankings may be used with the presently described processes and systems. The displays may also provide a rating of the vendors or drugs by indicators that show an ordinal ranking. These can include number indicators, letter indicators or color coded indicators (e.g., a heat map).

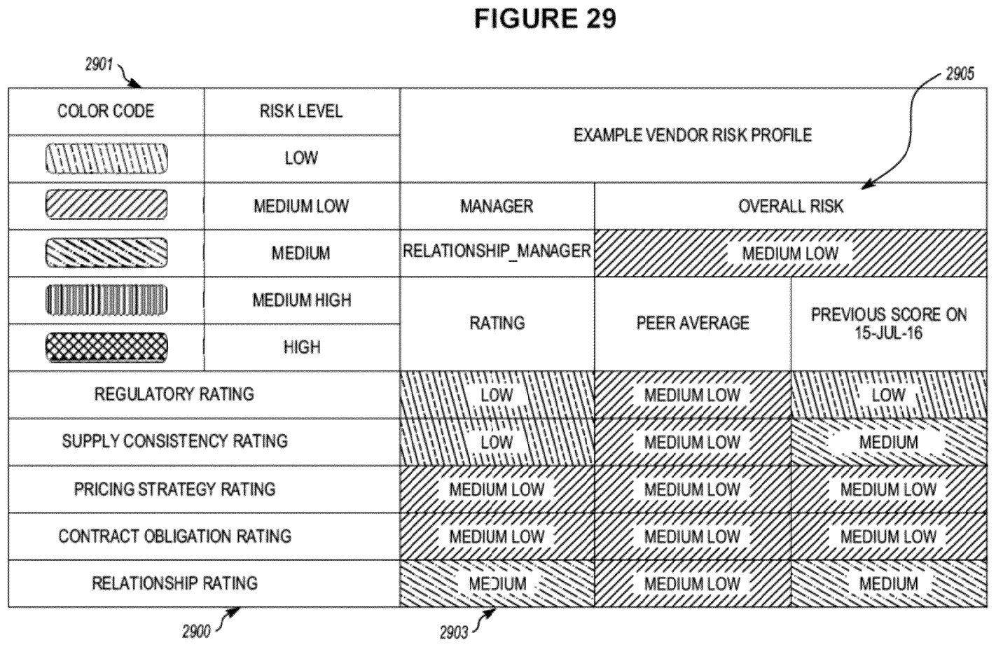

FIG. 29 shows a vendor risk scorecard view 2900 that can be shown on a display, according to an example embodiment, for a vendor. The view 2900 shows data relating to the overall risks that have been calculated in a single view. This risks can be the second level risks as described herein. The view 2900 may include a legend 2901 that relates the indicia, e.g., color coding, cross hatching fonts or other indicia to the risk as determined herein. In an example, a first color (e.g., green) is the indicia corresponding to a low risk level. In an example, a second color (e.g., blue) is the indicia corresponding to a medium-low risk level. In an example, a third color (e.g., yellow) is the indicia corresponding to a medium risk level. In an example, a fourth color (e.g., orange) is the indicia corresponding to a high-medium risk level. In an example, a fifth color (e.g., red) is the indicia corresponding to a high risk level. The tiers of risk allow the risk to be displayed with the higher levels of risk being the ones in need of attention. The risks are evaluated and displayed in a grid 2903 with indicia for regulatory rating, supply consistency rating, pricing strategy rating, contract obligation rating and relationship rating. A quantitative risk result can also be shown in the appropriate grid cell. The illustrated view 2900 show the current rating of risk, the peer average rating of risk and the previous rating of risk. The overall risk is also displayed at 2905. This view 2900 provides an easy to read and act upon display of risk for a particular vendor.

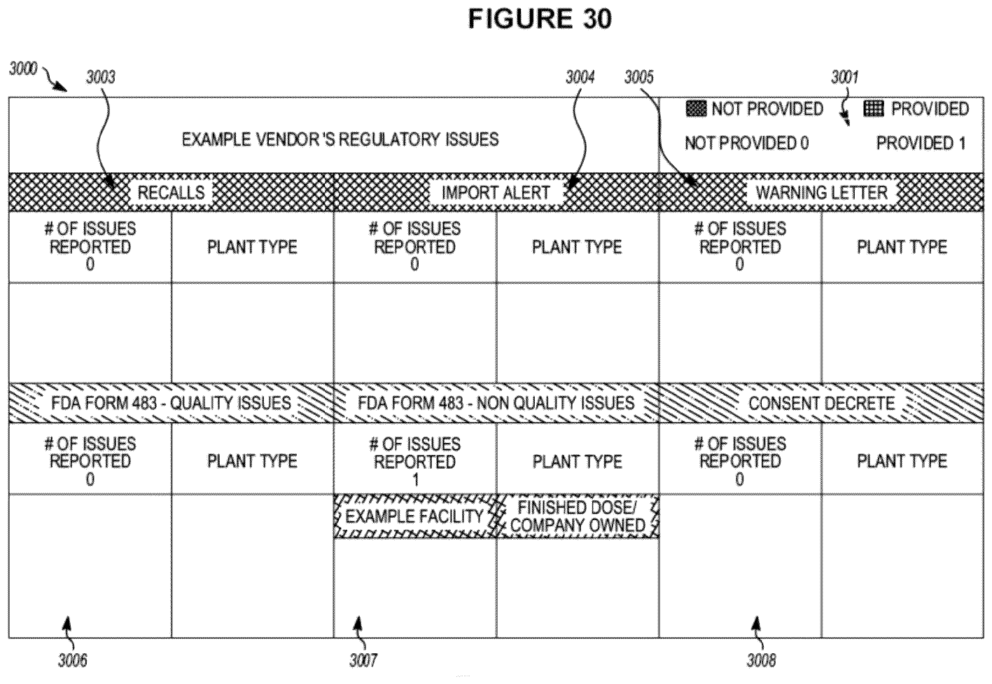

FIG. 30 is a vendor risk scorecard view 3000 that can be shown on a display, according to an example embodiment. The view 3000 shows the risks associated with a facility and may pertain to the vendor's regulatory issues for the vendor's facilities. The total number of regulatory issues is shown at 3001 and whether they were provided or not. The recalls with the number of issues and the plant type are shown at 3003. The import alerts with the number of issues and the plant type are shown at 3004. The warning letters with the number of issues and the plant type are shown at 3005. The quality issues with the number of issues and the plant type are shown at 3006. The non-quality issues with the number of issues and the plant type are shown at 3007. The consent decrees with the number of issues and the plant type are shown at 3008. These are the types of issues that result in greater risk and should be accounted for when setting contract terms or deciding whether to continue or end the relationship with a particular vendor in view of these regulatory issues. The data shown in view 3000 can be used in evaluating risk.

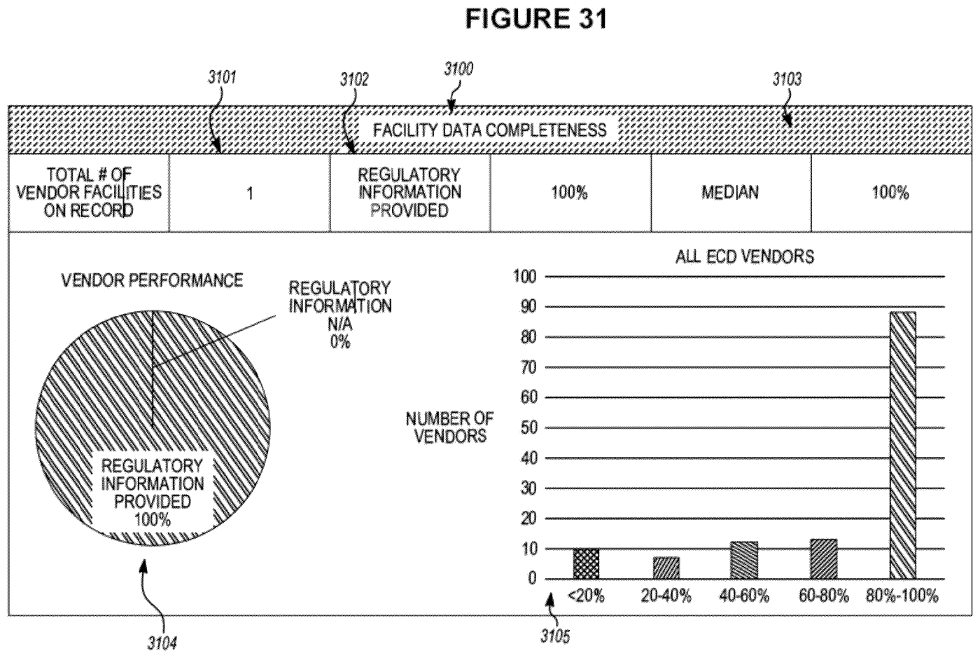

FIG. 31 is a facility data completeness view 3100 that can be shown on a display. Display box 3101 shows the total number of facilities in the system for a particular vendor. Display box 3102 shows the regulatory information provide, e.g., as a ratio of percentage of all of the facilities for the vendor. Display box 3103 shows the median of replies for similar vendors. Graph 3104 shows the regulatory information provided in a graphical format. A graph 3105 shows the vendors tiered by compliance, here in quintiles. Other groupings can be used, e.g., quartiles. The facility data completeness view 3100 provides a visual representation of how compliant the vendor is about providing data as requested or required.

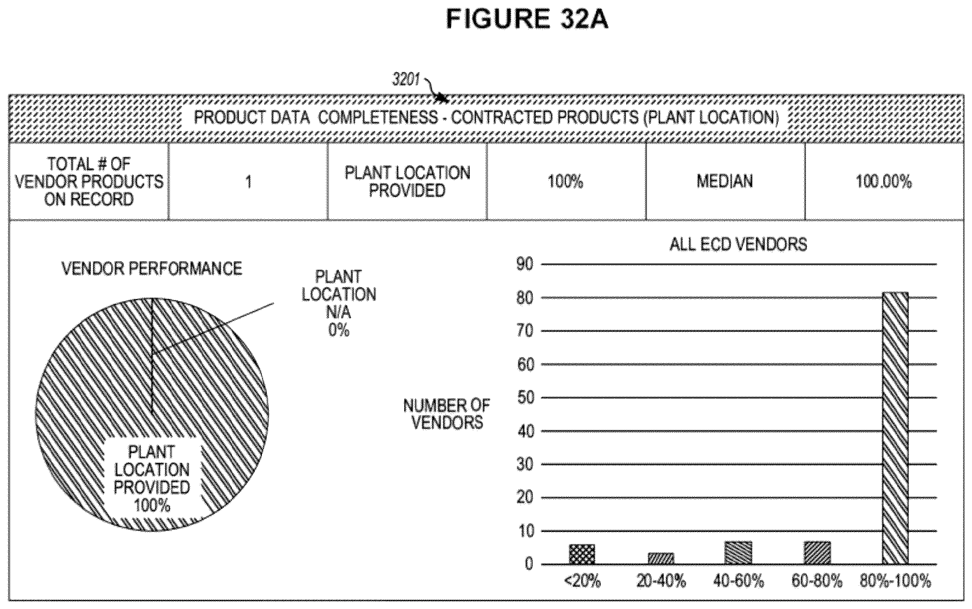

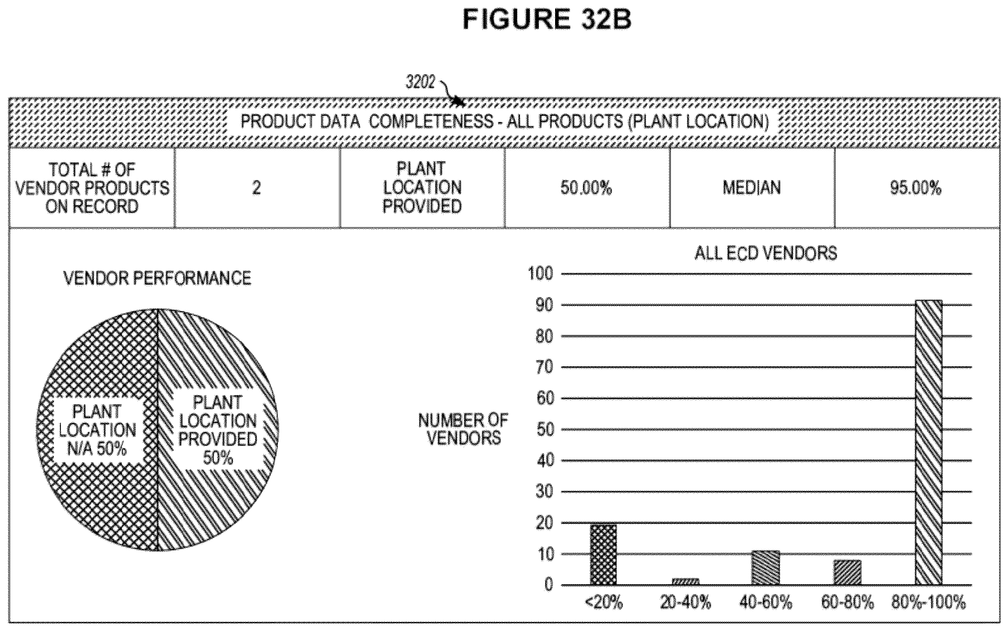

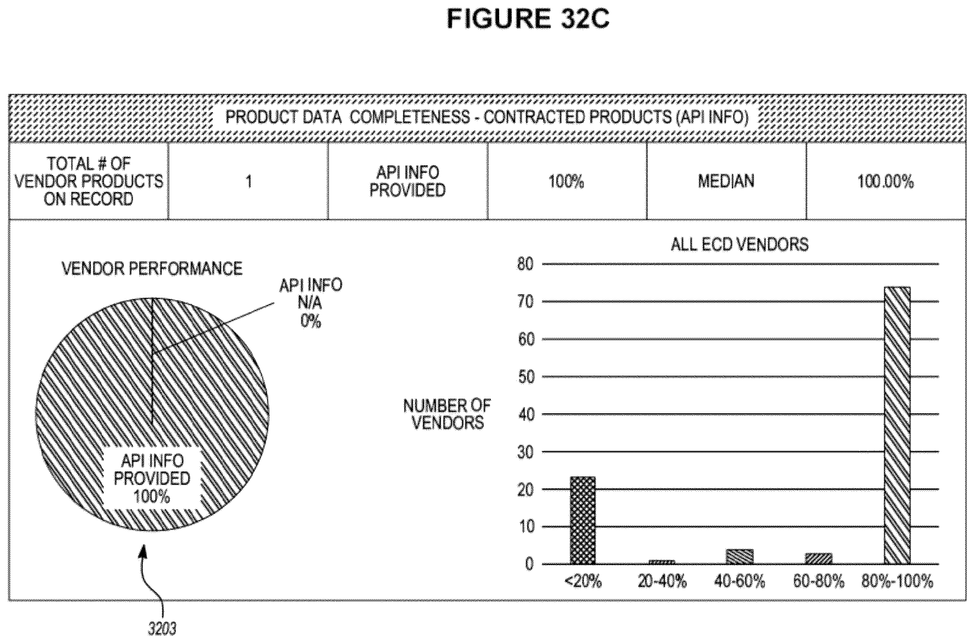

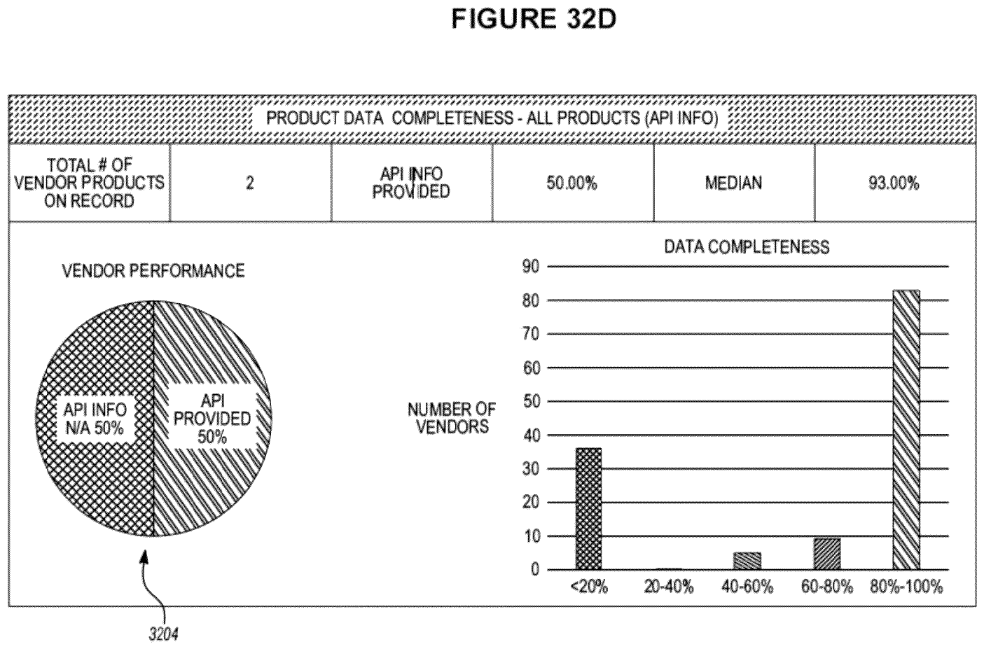

FIGS. 32A-D are view of a display showing multiple vendor risk scorecards 3201, 3202, 3203 and 3204, according to example embodiments. These views are similar in layout to view 3100 but are for different data associated with risk. Each view has data boxes and two graphs. View 3201 is the contracted products and plant location provided view. View 3201 shows the information related to whether the location of a plant that is used to manufacture a particular drug is provided by the vendor. View 3202 is the product data completeness for all products by plant location. View 3202 shows the information related to whether the location of a plant for all products of vendor are provided. View 3203 is the contracted products, active pharmaceutical ingredient (API) view. View 3204 shows a product completeness view for all products as it relates to the API. These views can provide a snapshot of how vendor is performing and how it is performing relative to other vendors. This data can be shared on a drug vendor device 113, a participant device 112 or the user device 110. These views can be generated by the analysis device 102.

The present disclosure includes embodiments that allow an organization being rated by a ratings agency, e.g., PBM, generic drug supplier, generic drug organization, an industry coalition or a consumer organization, to see the risks for disruption in a supply of a drug. The risk ratings that do not meet thresholds can be indicated or highlighted to bring these to the notice of the organization for corrective action. The present disclosure also provides a predictive feature that shows the organization the effects of a change in input data relating to a drug, a vendor, or regulation. The input data's effect on the rankings can be weighted when computing the ratings.

The present disclosure includes embodiments where visual indications show where a risk rating lies, e.g., on a spectrum or above or below a threshold value. It will be within the scope of the present disclosure to provide other indications of a risk rating not meeting a threshold. The present systems may provide an electronic indication, e.g., an email or a text message, to a responsible party to address the risk, e.g., explore other suppliers or other equivalent drugs. The electronic communication can include a link to the display that shows the visual indication. In another embodiment, the electronic communication can include the images of the risk ratings, e.g., a simplified subset of the displays 600-800.

It will be appreciated that the present disclosure is particularly suited for generic or off-patent drugs that are available by prescription. It will further be appreciated that the present disclosure can also be used to asses risk for over-the-counter medications and its suppliers.

The present systems and methods may provide a means to congeal a large amount of disparate data, from multiple sources, all relating to drug supply evaluation of risk. The disparate data can be interpreted using the rules described herein to provide a visual representation of risk on a display. The data can be weighted to provide a meaningful display output of risk.

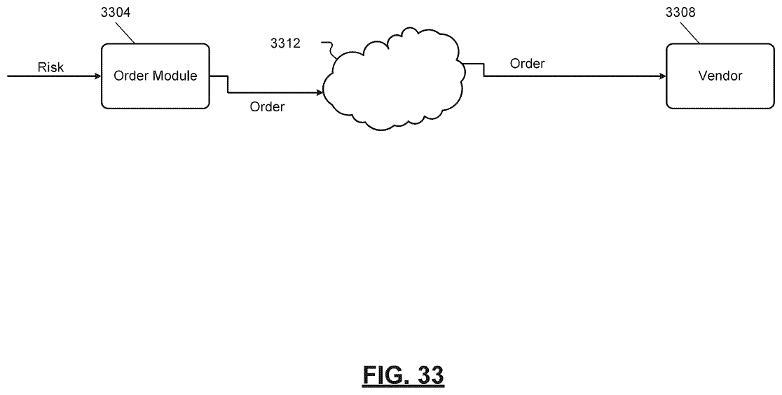

FIG. 33 is a functional block diagram of an example ordering system. An order module 3304 receives the risks discussed above, such as with respect to FIGS. 23A-23D. The order module 3304 may be, for example, part of the analysis device, another device, or independently implemented. When a risk is greater than a predetermined value, the order module 3304 automatically places an order to purchase a quantity of a drug with a vendor or manufacturer 3308 of the drug. The predetermined value may be calibratable. Additionally or alternatively, the order module 3304 may automatically place an order to purchase a quantity of a drug with the vendor 3308 of the drug when a risk increases by at least a predetermined value. The order module 3304 may transmit the order to the vendor 3308 via a network 3312 in various implementations. In various implementations, the order may be in the form of a purchase order. Payment may be included with the order in various implementations.

The order module 3304 determines the quantity of the drug to order based on various parameters, such as a predetermined minimum order quantity of the drug from the vendor 3308, a demand lead time for the drug, a re-order amount for the drug, a number of purchase orders per year from the vendor 3312 for the drug, and a target order quantity. The order module 3304 may determine the quantity of the drug to order using one or more lookup tables and/or equations that relate one or more of predetermined minimum order quantities, demand lead times, re-order amounts, numbers of purchase orders per year, and target order quantities to quantities to order.

The predetermined minimum order quantity may be set by the vendor 3308 of the drug and correspond to a minimum quantity of the drug allowed in a single order from the vendor 3308. The order module 3304 may determine the demand lead time, for example, based on a lead time for the drug and a daily volume of orders of the drug filled per day by the pharmacy. The order module 3304 may determine the demand lead time, for example, using an equation or a lookup table that relates lead times and daily volumes to demand lead times. For example, the order module 3304 may set the demand lead time based on or equal to the lead time multiplied by the daily volume (lead time*daily volume). The demand lead time corresponds to a number of units of the drug.

The order module 3304 may determine the re-order amount based on a predetermined safety stock amount of the drug and the demand lead time (amount) of the drug. The order module 3304 may determine the re-order amount, for example, using an equation or a lookup table that relates predetermined safety stock amounts and demand lead times to re-order amounts. For example, the order module 3304 may set the re-order amount based on or equal to the predetermined safety stock amount plus the demand lead time.

The order module 3304 may determine the number of purchase orders per year based on the number of days in a year (365 or 366 for leap years) and a lead time of the vendor 3008. The order module 3304 may determine the number of purchase orders, for example, using an equation or a lookup table that relates days in a year and lead times to numbers of purchase orders per year. For example, the order module 3304 may set the number of purchase orders per year based on or equal to 365 divided by the lead time of the vendor 3308.

The order module 3304 may determine the target order quantity, for example, based on an order quantity and the risk value. The order module 3304 may set the target order quantity, for example, using one of an equation and a lookup table that relates order quantities and risk values to target order quantities. For example only, the order module 3304 may set the target order quantity based on or equal to the order quantity multiplied by a square root of the risk value.

The order module 3304 may, for example, determine the quantity to order based on the minimum order quantity for the drug and an additional quantity of the drug to be ordered per purchase order. The order module 3304 may set the quantity to order, for example, based on or equal to the minimum order quantity plus the additional quantity.

The order module 3304 may determine the additional quantity, for example, based on a variance amount of the drug and the number of purchase orders per year. For example, the order module 3304 may set the additional quantity based on or equal to the variance amount of the drug divided by the number of purchase orders for the drug per year.

The order module 3304 may determine the variance amount based on the a demand and target safety stock amount of the drug, a minimum order quantity per year given lead time amount of the drug, and a present amount of the drug in inventory. For example, the order module 3304 may set the variance amount based on or equal to the demand and target safety stock amount of the drug minus the minimum order quantity per year given the lead time amount of the drug minus the present amount of the drug in inventory.

The order module 3304 may determine the demand and target safety stock amount of the drug based on the annual volume of the drug sold by the pharmacy and the predetermined safety stock of the drug for the pharmacy. for example, the order module 3304 may set the demand and target safety stock amount of the drug based on or equal to the annual volume of the drug plus the predetermined safety stock of the drug.

The order module 3304 may set the minimum order quantity per year given lead time amount of the drug based on the minimum order quantity of the drug and the target number of purchase orders for the drug per year. For example, the order module 3304 may set the minimum order quantity per year given the lead time amount based on or equal to the minimum order quantity multiplied by the target number of purchase orders for the drug per year. The target number of purchase orders per year may be determined as described above or set to a predetermined value. The minimum order quantity of the drug may be a predetermined value and specified by the vendor 3308.

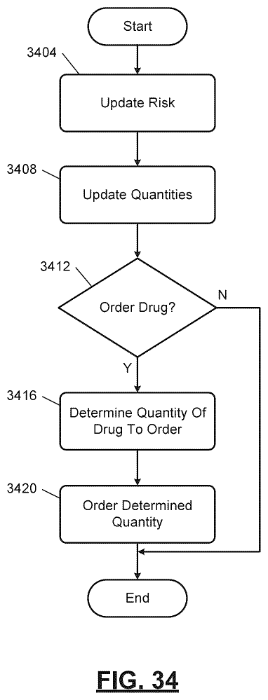

FIG. 34 is a flowchart depicting an example method of automatically ordering a drug based on risk associated with one of the drug and a vendor of the drug. Control may begin with 3404 where the risks are updated, such as described above with respect to FIGS. 23A-23D.

At 3408, the order module 3304 updates the quantities and other parameters discussed above with respect to FIG. 33. The quantities and other parameters may vary based on the risk. For example, a greater quantity may be desired to be ordered if a risk associated with the drug or a risk associated with the vendor of the drug increases, for example, to account for possible inability to obtain the drug in the future.

At 3412, the order module 3304 determines whether to place an order for the drug from the vendor 3308. If 3412 is true, control continues with 3416. If 3412 is false, the order module 3304 may not order the drug from the vendor 3308.

At 3416, the order module 3304 determines the quantity of the drug to order from the vendor 3308, such as described above with respect to FIG. 33. For example, the order module 3304 may determine the quantity of the drug to order based on or equal to the minimum order quantity of the drug from the vendor 3308 plus the additional amount.

At 3420, the order module 3304 automatically orders the quantity of the drug from the vendor 3308. For example, the order module 3304 may automatically generate a purchase order for the quantity of the drug from the vendor 3308. The order module 3304 may transmit the purchase order for the quantity of the drug to the vendor 3308, such as via the network 3312.

The present disclosure uses the term “risk” throughout. Risk may be the possibility of likelihood that something of value may be lost, e.g., a supply of a drug used as medicine. However, predicting risk is difficult in the field of drugs and generic drugs. The manufacture of drugs is spread throughout the world. There are too many variables with regard to risk for a person to accurately predict risk. The variables include business conditions, political conditions, supply of ingredients, conditions of manufacturing plants, weather, governmental regulation, supply/demand, health of a population and the like. Quickly these variables can escalate into the hundreds and even thousands for each drug, which can also number over ten thousand. Thus, as discovered by the present inventor, there is a need to develop a machine to compile and relate the factors. The present systems and methods use the factors to calculate multiple types of risk, e.g., on an organizational level or on a drug level. Moreover, a change in any factor, e.g., a given plant is shut down or a supply of an API is interrupted, will result in a real-time indication of risk related to any drug that is affected by the change in the factor. The present methods and systems can calculate this effect across all drugs and related organizations. Appropriate personnel can receive notifications of any potential problems in essentially real-time.

The term “based on” or using, as used herein, reflects an open-ended term that can reflect others elements beyond those explicitly recited.

Certain systems, apparatus, applications or processes are described herein as including a number of modules. A module may be a unit of distinct functionality that may be presented in software, hardware, or combinations thereof. When the functionality of a module is performed in any part through software, the module includes a computer-readable medium. The modules may be regarded as being communicatively coupled. A module may be a storage location in a database on a computer server, e.g., in an SQL database. The weights can be stored in calculated fields in the SQL database. The outputs for display may also be stored in the database and later transmitted to a display according to access rights and rules that filter the types of outputs to various authorized devices.

The inventive subject matter may be represented in a variety of different embodiments of which there are many possible permutations.

The ratings as determined herein may be part of a group purchasing organization and/or a private label distributor or calculated by a device associated with the GPO and/or private label distributor. The ratings may not be part of a pharmacy benefits manager in some example embodiments. The GPO and/or private label distributor can combine claims data and additional data, e.g., public data, to determine the ratings.

While the description generally reflects that certain methods, systems, and/or operations are performed by the participant and associated device, the methods, systems, and/or operations may additionally or alternatively be performed by the a participant and an associated device and/or the exchange and associated device.

Thus, methods and systems for risk assessment and adjustment have been described. Although embodiments of the present invention have been described with reference to specific example embodiments, it will be evident that various modifications and changes may be made to these embodiments without departing from the broader spirit and scope of the embodiments of the invention. Accordingly, the specification and drawings are to be regarded in an illustrative rather than a restrictive sense.

The present methods and systems can track a large number of drugs, e.g. or more, which could not be tracked to determine risks to a supply of all of those drugs as the supply of each of those drugs includes factors relating to the manufacturing location, supply of materials to manufacture and geo-political factors, and the like.

The methods described herein do not have to be executed in the order described, or in any particular order. Moreover, various activities described with respect to the methods identified herein can be executed in serial or parallel fashion. Although “End” blocks are shown in the flowcharts, the methods may be performed continuously.

The embodiments of the present disclosure generally provide for a plurality of circuits or other electrical devices, which can be used in units, modules, systems, and sub-systems and the like. All references to such and the functionality provided by each are not intended to be limited to encompassing only what is illustrated and described herein. While particular labels may be assigned to the various circuits or other electrical devices disclosed, such labels are not intended to limit the scope of operation for the circuits and the other electrical devices. Such circuits and other electrical devices may be combined with each other and/or separated in any manner based on the particular type of electrical/operational implementation that is desired. It is recognized that any circuit or other electrical device disclosed herein may include any number of microprocessors, discrete circuit components, integrated circuits, memory devices (e.g., FLASH, random access memory (RAM), read only memory (ROM), electrically programmable read only memory (EPROM), electrically erasable programmable read only memory (EEPROM), or other suitable variants thereof, etc.) and instructions (e.g., software, etc.) which co-act with one another to perform operation(s) disclosed herein. In addition, any one or more than one electric devices may be configured to execute a computer-program that is embodied in a computer readable medium that is programmed to perform any number of the functions and features as disclosed. The computer readable medium may be non-transitory or in any form readable by a machine or electrical component.

In this application, including the definitions below, the term “module” or the term “controller” may be replaced with the term “circuit.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

The term code, as used above, may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks, flowchart components, and other elements described above serve as software specifications, which can be translated into the computer programs by the routine work of a skilled technician or programmer.

The computer programs include processor-executable instructions that are stored on at least one non-transitory, tangible computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation) (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

The Abstract of the Disclosure is provided to comply with 37 C.F.R. § 1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus, the following claims are hereby incorporated into the Detailed Description, with each claim standing on its own as a separate embodiment.

What is claimed is:

1. A system, comprising:
a network;
a governmental regulatory computing device;
a user computing device;
a participant computing device; and
an analysis computing device including memory and a processor configured to:
communicate with the governmental regulatory computing device via the network;
obtain, from the governmental regulatory computing device, data regarding at least one of a drug manufacturer that is subject to an exclusion order and a drug that is subject to an exclusion order; and
determine a risk associated with at least one of the drug and the drug manufacturer based on the data obtained from the governmental regulatory computing device,
wherein the participant computing device is configured to transmit a predicted demand for the at least one of the drug and the drug manufacturer to the analysis computing device via the network,
wherein the analysis computing device is configured to determine the risk associated with the at least one of the drug and the drug manufacturer further based on the predicted demand for the at least one of the drug and the drug manufacturer,
wherein the user computing device is configured to transmit a query to the analysis computing device over the network, the query requesting the risk associated with the at least one of the drug and the drug manufacturer,
wherein the analysis computing device is configured to transmit the risk of the at least one of the drug and the drug manufacturer to the user computing device via the network,
wherein the user computing device is configured to:
generate a report regarding the at least one of the drug and the drug manufacturer and include the risk in the report: and
display the report including the risk and the at least one of the drug and the drug manufacturer on a display, and
wherein the analysis computing device is further configured to, based on the risk, selectively automatically order a quantity of the drug from the drug manufacturer.

2. A method of ordering a drug from a vendor of the drug, the method comprising:
by one or more processors, using a first application programming interface (API), obtaining first level risk values associated with the vendor of the drug from two or more external data sources, the first level risk values representing real world quantities used to evaluate risk;
by the one or more processors, retrieving first predetermined weight values associated with the first level risk values, respectively, from a SQL database;
by the one or more processors, applying the first predetermined weight values to the first level risk values to obtain weighted first level risk values, respectively;
by the one or more processors, calculating second level risk values associated with the vendor based on the weighted first level risk values;
by the one or more processors, retrieving second predetermined weight values associated with the second level risk values, respectively, from the SQL database;
by the one or more processors, applying the second predetermined weight values to the second level risk values to obtain weighted second level risk values, respectively;
by the one or more processors, calculating third level risk values associated with the vendor based on the weighted second level risk values;
by the one or more processors, retrieving third predetermined weight values associated with the third level risk values, respectively, from the SQL database;
by the one or more processors, applying the third predetermined weight values to the third level risk values to obtain weighted third level risk values, respectively;
by the one or more processors, calculating an overall risk value associated with the vendor based the weighted third level risk values;
by the one or more processors, in response to receipt of user input for the overall risk value for the vendor of the drug, displaying a visual indicator of the overall risk value of the vendor of the drug on a display,
by the one or more processors, changing one of the first, second, and third level risk values and recalculating the overall risk value using the changed one of the first, second, and third level risk values via retrieval using a second API;
by the one or more processors, visually indicating on the display all of the first, second, and third level risk values that drop below a first threshold in response to the changing of the one of the first, second, and third level risk values;
by the one or more processors, transmitting an electronic alert to an associated device when one of the first, second, and third level risk values exceeds a second threshold;
by the one or more processors, visually changing a risk indicator as at least one of the first level, second level, third level, and overall risk values increases; and
by the one or more processors, based on the overall risk value, selectively ordering a quantity of the drug from the vendor.

3. The method of claim 2 wherein calculating the second level risk values includes setting the second level risk values equal to sums of ones of the weighted first level risk values.

4. The method of claim 3 wherein calculating the third level risk values includes setting the third level risk values equal to sums of ones of the weighted second level risk values.

5. The method of claim 4 wherein calculating the overall risk value includes setting the overall risk value equal to a sum of the weighted third level risk values.

6. The method of claim 2 wherein one of the first level risk values is a regulatory compliance risk value, and
wherein the one of the first predetermined weighting values associated with the regulatory compliance risk value is greater than all of the other ones of the first predetermined weighting values.

7. The method of claim 2 wherein obtaining the first level risk values includes obtaining at least one of the first level risk values from an electronic drug vendor query.

8. The method of claim 2 wherein obtaining the first level risk values includes storing at least one of the first level risk values in response to user input regarding the at least one of the first level risk values.

9. The method of claim 2 wherein obtaining the first level risk values includes retrieving at least one of the first level risk values from a database.

10. The method of claim 2 wherein the first level risk values are each a value between 0 and 100, and the first predetermined weighting values are values between 0 and 100 percent.

11. The method of claim 10 wherein the second level risk values are each a value between 0 and 100, and the second predetermined weighting values are values between 0 and 100 percent.

12. The method of claim 11 wherein the third level risk values are each a value between 0 and 100, and the third predetermined weighting values are values between 0 and 100 percent.

13. The method of claim 2 wherein the first level risk values include at least 10 different first level risk values associated with the vendor of the drug.

14. The method of claim 13 wherein the second level risk values include at least different second level risk values.

15. The method of claim 14 wherein the third level risk values include 2 different third level risk values.

16. A system for ordering a drug from a vendor of the drug, the system including:
memory including instructions; and
one or more processors configured to execute the instructions to:
obtain first level risk values associated with the vendor of the drug using a first application programming interface (API), the first level risk values representing real world quantities used to evaluate risk;
retrieve first predetermined weight values associated with the first level risk values, respectively, from a SQL database;
apply the first predetermined weight values to the first level risk values to obtain weighted first level risk values, respectively;
calculate second level risk values associated with the vendor based on the weighted first level risk values;
retrieve second predetermined weight values associated with the second level risk values, respectively, from the SQL database;
apply the second predetermined weight values to the second level risk values to obtain weighted second level risk values, respectively;
calculate third level risk values associated with the vendor based on the weighted second level risk values;
retrieve third predetermined weight values associated with the third level risk values, respectively, from the SQL database;
apply the third predetermined weight values to the third level risk values to obtain weighted third level risk values, respectively;
calculate an overall risk value associated with the vendor based the weighted third level risk values;
in response to receipt of user input for the overall risk value for the vendor of the drug, display a visual indicator of the overall risk value of the vendor of the drug on a display;
change one of the first, second, and third level risk values and recalculate the overall risk value using the changed one of the first, second, and third level risk values via retrieval using a second API;
visually indicate on the display all of the first, second, and third risk values that drop below a first threshold in response to the changing of the one of the first, second, and third level risk values;
transmit an electronic alert to an associated device when one of the first, second, and third level risk values exceeds a second threshold;
visually change a risk indicator as at least one of the first level, second level, third level, and overall risk values increases; and
based on the overall risk value, selectively order a quantity of the drug from the vendor.

17. A method of ordering a drug, the method comprising:
by one or more processors, using a first application programming interface (API), obtaining first level risk values associated with the drug, the first level risk values representing real world quantities used to evaluate risk;
by the one or more processors, retrieving first predetermined weight values associated with the first level risk values, respectively, from a SQL database;
by the one or more processors, applying the first predetermined weight values to the first level risk values to obtain weighted first level risk values, respectively;
by the one or more processors, calculating second level risk values associated with the drug based on the weighted first level risk values;
by the one or more processors, retrieving second predetermined weight values associated with the second level risk values, respectively, from the SQL database;
by the one or more processors, applying the second predetermined weight values to the second level risk values to obtain weighted second level risk values, respectively;
by the one or more processors, calculating an overall risk value associated with the drug based the weighted second level risk values;
by the one or more processors, in response to receipt of user input for the overall risk value for the drug, displaying a visual indicator of the overall risk value for the drug on a display;
by the one or more processors, changing one of the first and second level risk values and recalculating the overall risk value using the changed one of the first and second level risk values via retrieval using a second API;
by the one or more processors, visually indicating on the display all of the first and second level risk values that drop below a first threshold in response to the changing of the one of the first and second level risk values;
by the one or more processors, transmitting an electronic alert to an associated device when one of the first and second risk values levels exceeds a second threshold; and
by the one or more processors, visually changing a risk indicator as at least one of the first level, second level, and overall risk values increases; and
by the one or more processors, based on the overall risk value for the drug, selectively ordering a quantity of the drug from a vendor of the drug.

18. The method of claim 17 wherein calculating the second level risk values includes setting the second level risk values equal to sums of ones of the weighted first level risk values.

19. The method of claim 18 wherein calculating the overall risk value includes setting the overall risk equal to a sum of the weighted second level risk values.

20. The method of claim 17 wherein obtaining the first level risk values includes obtaining at least one of the first level risk values from an electronic drug vendor query.

21. The method of claim 17 wherein obtaining the first level risk values includes storing at least one of the first level risk values in response to user input regarding the at least one of the first level risk values.

22. The method of claim 17 wherein obtaining the first level risk values includes retrieving at least one of the first level risk values from a database.

23. The method of claim 22 wherein the first level risk values are each a value between 0 and 100, and the first predetermined weighting values are values between 0 and 100 percent.

24. The method of claim 23 wherein the second level risk values are each a value between 0 and 100, and the second predetermined weighting values are values between 0 and 100 percent.

25. The method of claim 17 wherein the first level risk values include at least 10 different first level risk values associated with the drug.

26. The method of claim 25 wherein the second level risk values include 4 different second level risk values.

27. A method of determining a risk associated with a drug and a vendor of that drug, the method comprising:
by one or more processors, using a first application programming interface (API), obtaining first level risk values associated with the vendor of the drug, the first level risk values associated with the vendor of the drug representing real world quantities used to evaluate risk;
by the one or more processors, retrieving first predetermined weight values associated with the first level risk values associated with the vendor, respectively, from a SQL database;
by the one or more processors, applying the first predetermined weight values to the first level risk values associated with the vendor to obtain weighted first level risk values associated with the vendor, respectively;
by the one or more processors, calculating second level risk values associated with the vendor based on the weighted first level risk values associated with the vendor;
by the one or more processors, retrieving second predetermined weight values associated with the second level risk values associated with the vendor, respectively, from the SQL database;
by the one or more processors, applying the second predetermined weight values to the second level risk values associated with the vendor to obtain weighted second level risk values associated with the vendor, respectively;
by the one or more processors, calculating third level risk values associated with the vendor based on the weighted second level risk values associated with the vendor;
by the one or more processors, retrieving third predetermined weight values associated with the third level risk values associated with the vendor, respectively, from the SQL database;
by the one or more processors, applying the third predetermined weight values to the third level risk values associated with the vendor to obtain weighted third level risk values associated with the vendor, respectively;
by the one or more processors, calculating a first overall risk value associated with the vendor based the weighted third level risk values associated with the vendor;
by the one or more processors, using the first API, obtaining first level risk values associated with the drug, the first level risk values associated with the drug representing real world quantities used to evaluate risk;
by the one or more processors, retrieving fourth predetermined weight values associated with the first level risk values associated with the drug, respectively, from the SQL database;
by the one or more processors, applying the fourth predetermined weight values to the first level risk values associated with the drug to obtain weighted first level risk values associated with the drug, respectively;
by the one or more processors, calculating second level risk values associated with the drug based on the weighted first level risk values;
by the one or more processors, retrieving fifth predetermined weight values associated with the second level risk values associated with the drug, respectively, from the SQL database;
by the one or more processors, applying the fifth predetermined weight values to the second level risk values associated with the drug to obtain weighted second level risk values associated with the drug, respectively;
by the one or more processors, calculating a second overall risk value associated with the drug based the weighted second level risk values associated with the drug;
by the one or more processors, retrieving sixth and seventh predetermined weight values associated with the first and second overall risk values, respectively, from the SQL database;
by the one or more processors, applying the sixth and seventh predetermined weight values to the first and second overall risk values to obtain weighted first and second overall risk values, respectively;
by the one or more processors, calculating a third overall risk value associated with the vendor supplying the drug based on the weighted first and second overall risk values; and
by the one or more processors, in response to receipt of user input for the third overall risk value, displaying a visual indicator of the third overall risk value on a display;
by the one or more processors, changing one of the first, second, and third level risk values and recalculating the third overall risk value using the changed one of the first, second, and third level risk values via retrieval using a second API;
by the one or more processors, visually indicating on the display all of the first, second, and third level risk values that drop below a first threshold in response to the changing of the one of the first, second, and third level risk values;
by the one or more processors, transmitting an electronic alert to an associated device when one of the first, second, and third level risk values exceeds a second threshold;
by the one or more processors, visually changing a risk indicator as at least one of the first level risk values, the second level risk values, the third level risk values, the first overall risk value, the second overall risk value, and the third overall risk value increases; and
by the one or more processors, based on the third overall risk value associated with the vendor supplying the drug, selectively ordering a quantity of the drug from the vendor of the drug.

28. The method of claim 27 wherein calculating the third overall risk value includes setting the third overall risk value equal to a sum of the weighted first and second overall risk values.
